(19) United States
(12) Patent Application Publication
(10) Pub. No.: US 2014/0066365 A1
(43) Pub. Date: Mar. 6, 2014

(54) USE OF CALIXARENES ASSOCIATED WITH AN ANTIBIOTIC IN THE TREATMENT OF BACTERIAL INFECTIONS

(71) Applicants: Marion Grare, Toulouse (FR); Raphaël, Emmanuel Duval, Bioncourt (FR)

(72) Inventors: Marion Grare, Toulouse (FR); Raphaël, Emmanuel Duval, Bioncourt (FR)

(21) Appl. No.: 14/110,791

(22) PCT Filed: Apr. 11, 2012
(86) PCT No.: PCT/FR12/50790

§ 371 Date: Nov. 20, 2013

(30) Foreign Application Priority Data
Apr. 12, 2011 (FR) 11/53205

(51) Int. Cl. A61K 31/155; A61K 31/65; A61K 31/575; A61K 31/7036; A61K 31/665; A61K 31/43; A61K 31/431; A61K 31/496; A61K 38/12

(52) U.S. Cl. CPC A61K 31/155; A61K 31/496; A61K 31/65; A61K 31/575; A61K 38/12; A61K 31/665; A61K 31/43; A61K 31/431; A61K 31/7036
USPC 514/2.9; 514/254.11; 514/154; 514/171; 514/99; 514/199; 514/196; 514/37

(57) ABSTRACT

A product comprising at least one given antibiotic and a calixarene for use as medicament.

The present invention relates to the use of calixarenes with an antibiotic in the treatment of bacterial infections.

In the field of public health, the fight against community-acquired or nosocomial bacterial infections is always a subject of topicality and concern. In fact, bacteria are the microorganisms that are most often responsible for nosocomial infections (NI), with, in order of frequency: Escherichia coli (24.7%), Staphylococcus aureus (18.9%), Pseudomonas aeruginosa (10%) and Enterococcus spp. (6%) (RAISIN Enquiry 2006).

Certain bacteria involved in hospitals have a resistance or even a multi-resistance to the antibiotics and/or antiseptics routinely used. Multi-Resistant Bacteria are referred to as MRBs and Toto-Resistant Bacteria as TRBs. There can be mentioned for example MRSA (meticillin-resistant Staphylococcus aureus, with a resistance to all the β-Lactams), Enterobacteria carrying ESBL (Extended spectrum β-Lactamase) or also GRE (glycopeptide resistant Enterococcus spp.). Currently, 64% of the Staphylococcus aureus isolated during NI are meticillin-resistant (RAISIN Enquiry 2006). The problem is that the bacteria often carry several resistance mechanisms, inducing a resistance to numerous families of antibiotics: β-lactams, aminoglycosides, fluoroquinolones or macrolides etc. Moreover, this resistance to antibiotics is often associated with a resistance to the antiseptics used in the hospital environment for combating the dissemination of nosocomial infections.

The resistance of a bacterial strain to an antibiotic can be a natural resistance (characteristic of all the strains of the same species). It can be also acquired (characteristic of certain strains within a species); it then results from a modification of the gene pool of these bacteria. This type of genetic modification can confer on a bacterial strain concerned a mechanism of resistance to an antibiotic, to a family of antibiotics or to several families of antibiotics.

Fundamental research into the mechanisms used by the bacteria and the epidemiological data are currently giving rise to doubts about the possibility of eradicating these MRBs in the future. Therefore, it is no longer certain that the currently available antibiotics make it possible to control the problem over the long term. If the availability of novel antibiotics has until now made it possible to respond to each form of bacterial resistance, this approach now faces many limitations, as no new class of antibiotics has been developed for twenty five years (Boucher et al. CID, 2009). Few new antibiotics have been marketed since the start of the 90s. Among these new antibiotics, only linezolid and daptomycin have an innovative mechanism of action, but are reserved for quite specific and active applications only on Gram-positive bacteria. Moreover, they have a significant toxicity (haematological and medullar toxicity in the case of linezolid and eosinophilic pneumopathies in the case of daptomycin), which restricts their use.

However, very shortly after they were marketed, bacterial resistances appeared. Thus, by way of example, the following cases can be mentioned: linezolid, daptomycin, quinupristin-dalfopristin, or tigecycline, including in bacteria that were multi-resistant to begin with.

By using an innovative concept linking supramolecular chemistry with targeting and disorganization of the bacterial wall, a novel family of antibacterial compounds, in particular para-guanidinoethylcalix[4]arene, hereafter designated Cx1, has been developed recently. This family of compounds have antibacterial properties against different bacteria involved in nosocomial and/or community-acquired infections.

The publication by Grare et al. (J. Antimicrob. Chemother. 60 (2007), 575-581) describes that Cx1 has an antibacterial activity on bacteria which are resistant or not resistant to antibiotics.

In the publication by Grare et al. (Clin. Microbiol. Infect. 16 (2010), 432-438), the antibacterial activity of Cx1 is compared to that of hexamidine and chlorhexidine, two antiseptics which are very commonly used in human therapeutics, over a whole series of clinical isolates: MDR (“multidrug resistant”), XDR (“extended drug resistant”), even PDR (“pan-drug resistant”).

The article by Grare et al. (Pathologie Biologie 58 (2010), 46-51) describes that Cx1, as a cationic antibacterial, interacts with the bacterial wall, leading in the end to a loss of membrane integrity.

Nevertheless, faced with the threat of the emergence of PDR bacteria, it remains a matter of absolute urgency to be able to have available, novel antibacterial compounds having innovative mechanisms of action, for treating patients infected with this type of bacteria; and/or novel means making treatment with the antibiotics normally used in anti-infectious therapeutics, again accessible to these patients.

An aspect of the present invention is to provide novel antibacterial products.

Another aspect of the invention is to supply novel antibacterial compositions combining calixarenes and antibiotics.

The present invention is based on an unexpected fact noted by the Inventors, during evaluation of the antibacterial activity of para-guanidinoethylcalix[4]arene, hereafter designated Cx1. This molecule makes it possible to reduce the MIC (Minimum Inhibitory Concentration) of an antibiotic to which a bacterial strain has a resistance.

In other words, on the one hand, Cx1 makes it possible to confer de novo a certain level of susceptibility to (an) antibiotic(s) in a bacterial strain having an acquired resistance to said antibiotic(s), and on the other hand, Cx1 is also capable of conferring a susceptibility to (an) antibiotic(s) in a bacterial strain having a natural resistance to said antibiotic(s).

In concrete terms, in the clinical context, treatment with Cx1 in combination with at least one antibiotic makes it possible to reduce the dose of the latter in the context of the treatment of an infection with a bacterium resistant to said antibiotic, and/or to make the treatment with said antibiotic effective in patients infected with at least one bacterial strain resistant to said antibiotic.

The present invention proposes a product comprising at least one given antibiotic and a calixarene represented by Formula I below:

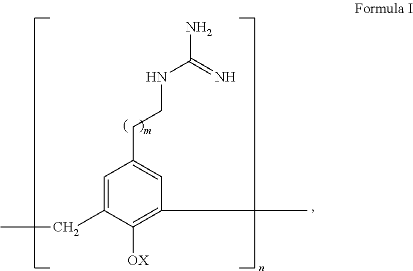

in which:

(i) n=an integer from 4 to 16,

(ii) m=an integer from 1 to 10,

(iii) X is chosen from:
a hydrogen,
an alkyl group, the number of carbons being from 1 to 20, in particular from 1 to 10,
a halogen chosen from Cl, Br, I, or
an amphiphilic group chosen from an anionic group, such as the carboxylates —RCO₂—, the sulphates —RSO₄—, the sulphonates —RSO₃—, a cationic group, such as RNH₃′, in which R is an alkyl group, the number of carbons being from 1 to 20, in particular from 1 to 10,
   for its use as medicament.

In a particular embodiment, the product according to the invention comprises a given antibiotic and a calixarene represented by Formula I, in which n=4, said calixarene represented by Formula I(1) below:

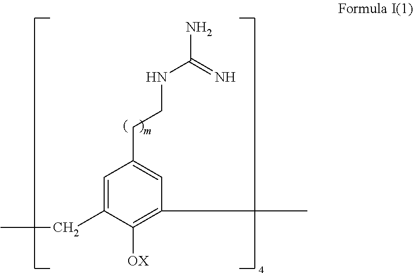

m and X having the meanings indicated above.

In another particular embodiment, the product according to the invention comprises a given antibiotic and a calixarene represented by Formula I, in which m=1, said calixarene represented by Formula I(2) below:

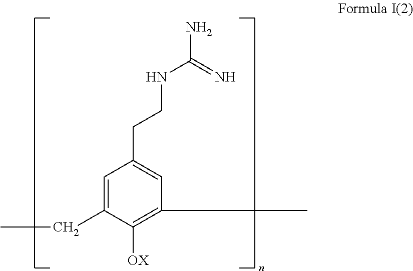

n and X having the meanings indicated above.

According to a particular embodiment, the product according to the invention comprises a given antibiotic and a calixarene represented by Formula I, in which X is a hydrogen, said calixarene represented by Formula I(3) below:

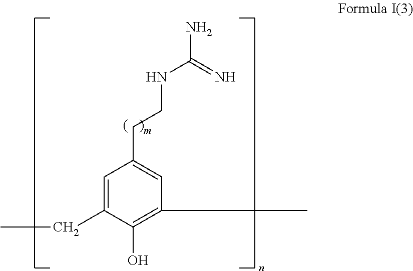

m and n having the meanings indicated above.

In an advantageous embodiment, the present invention relates to a product for use as medicament, said product comprising a given antibiotic and a calixarene represented by Formula I, in which n=4, m=1 and X is a hydrogen, said calixarene represented by Formula II below:

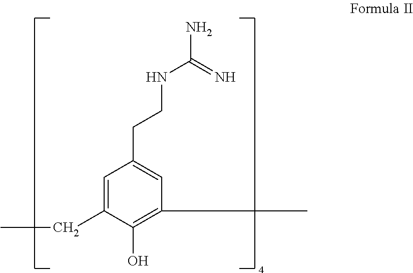

The molecule represented by Formula II is para-guanidinoethylcalix[4]arene, designated Cx1 in the present invention.

The three-dimensional structure of the above-mentioned molecule is illustrated below.

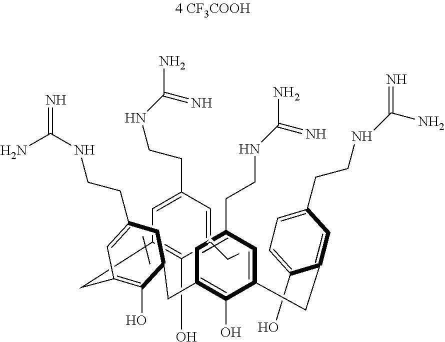

Cx1 can be synthesized according to the process described in Mourer et al. (Bioorganic & Medicinal Chemistry Letter 16 (2006) 2960-2963).

The calixarene according to the invention can be as described above, or a salt of a physiologically acceptable acid derived from a compound of Formula (I) such as a hydrochloride, a formate, a trifluoroacetate or an oxalate (HOOCCOOH).

The expression “salt of a physiologically acceptable acid” signifies a derivative of a compound of Formula I, obtained by the reaction of an inorganic acid or an organic acid, with a compound of Formula I.

Examples of inorganic acids making it possible to obtain physiologically acceptable salts include, but are not limited to, hydrochloric acid, hydrobromic acid, nitric acid, carbonic acid, formic acid, monohydrogen carbonic acid, phosphoric acid, monohydrogen phosphoric acid, dihydrogen phosphoric acid, perchloric acid, sulphuric acid, monohydrogen sulphuric acid, hydriodic acid.

Examples of organic acids making it possible to obtain physiologically acceptable salts include, but are not limited to, acetic acid, lactic acid, propionic acid, butyric acid, isobutyric acid, palmic acid, maleic acid, glutamic acid, hydroxymaleic acid, malonic acid, benzoic acid, succinic acid, glycolic acid, suberic acid, fumaric acid, mandelic acid, phthalic acid, salicylic acid, benzenesulphonic acid, p-toluenesulphonic acid, citric acid, tartaric acid, methanesulphonic acid, hydroxynaphthoic acid.

The salts of amino acids, such as the arginates and their equivalents are also included as well as the salts of organic acids such as glucuronic acid or galacturonic acid and their equivalents (see, for example, Berge et al., “Pharmaceutical Salts”, Journal of Pharmaceutical Science, 1977, 66, 1-19).

The given antibiotic utilized in the aforementioned product for its use as medicament according to the invention can be chosen from the β-lactams, the aminoglycosides, fluoroquinolones, fosfomycin, colimycin, rifampicin, tigecycline or fusidic acid, and more particularly from the group comprising imipenem, piperacillin-tazobactam, penicillin G, cefotaxime, ceftazidime, tobramycin, gentamicin, ciprofloxacin, rifampicin, fosfomycin, colimycin, streptomycin, ticarcillin-clavulanic acid, tigecycline or fusidic acid.

In a particularly advantageous embodiment, the product according to the invention comprises a calixarene represented by Formula II (Cx1) and tigecycline.

In another particularly advantageous embodiment, the product according to the invention comprises a calixarene represented by Formula II (Cx1) and fusidic acid.

In another particularly advantageous embodiment, the product according to the invention comprises a calixarene represented by Formula II (Cx1) and fosfomycin.

In another particularly advantageous embodiment, the product according to the invention comprises a calixarene represented by Formula II (Cx1) and penicillin.

In another particularly advantageous embodiment, the product according to the invention comprises a calixarene represented by Formula II (Cx1) and imipenem.

In another particularly advantageous embodiment, the product according to the invention comprises a calixarene represented by Formula II (Cx1) and gentamicin.

In another particularly advantageous embodiment, the product according to the invention comprises a calixarene represented by Formula II (Cx1) and cefotaxime.

In another particularly advantageous embodiment, the product according to the invention comprises a calixarene represented by Formula II (Cx1) and rifampicin.

In another particularly advantageous embodiment, the product according to the invention comprises a calixarene represented by Formula II (Cx1) and piperacillin-tazobactam.

In another particularly advantageous embodiment, the product according to the invention comprises a calixarene represented by Formula II (Cx1) and ciprofloxacin.

In another particularly advantageous embodiment, the product according to the invention comprises a calixarene represented by Formula II (Cx1) and ceftazidime.

In another particularly advantageous embodiment, the product according to the invention comprises a calixarene represented by Formula II (Cx1) and colimycin.

In another particularly advantageous embodiment, the product according to the invention comprises a calixarene represented by Formula II (Cx1) and ticarcillin-clavulanic acid.

In another particularly advantageous embodiment, the product according to the invention comprises a calixarene represented by Formula II (Cx1) and tobramycin.

In such a composition according to the invention, the calixarene represented by Formula II (Cx1) and a given antibiotic can be physically mixed together in a single end product.

The calixarene represented by Formula II (Cx1) and such a given antibiotic can also be present in the form of a single end product, but physically separated. For example, the Cx1 and the given antibiotic can be present respectively in two separate compartments of a capsule.

Another aspect of the invention relates to a product as described above for its use as medicament in the treatment of pathologies involving a bacterial strain having a resistance to at least one defined antibiotic.

In an advantageous embodiment, the invention relates to a product as described above for its use in the treatment of pathologies involving a resistant bacterial strain from Escherichia coli, Pseudomonas aeruginosa, and Staphylococcus aureus, more particularly a resistant bacterial strain chosen from:
a wild-type strain of Staphylococcus aureus
a strain of meticillin-resistant Staphylococcus aureus (MRSA) without associated resistance,
a strain of MRSA having a resistance to the aminoglycosides and fluoroquinolones,
a strain of MRSA having a resistance to the aminoglycosides, fluoroquinolones, macrolides-lincosamides-synergystins and ofloxacin,
a wild-type strain of Escherichia coli
a penicillinase-producing strain of Escherichia coli, without associated resistance,
an ESBL (Extended spectrum β-Lactamase)-producing strain of Escherichia coli, having an associated resistance to the aminoglycosides, rifampicin and the trimethoprime-sulphamethoxazole combination,
a cephalosporinase-hyperproducing strain of Escherichia coli having an associated resistance to the aminoglycosides, quinolones and the trimethoprime-sulphamethoxazole combination,
a wild-type strain of Pseudomonas aeruginosa
a strain of Pseudomonas aeruginosa having a resistance to the β-lactams, the trimethoprime-sulphamethoxazole combination and fosfomycin,
a strain of Pseudomonas aeruginosa having a resistance to the β-lactams (including the carbapenems), the aminoglycosides, the trimethoprime-sulphamethoxazole combination and ciprofloxacin,
a mucoid strain of Pseudomonas aeruginosa having a resistance to rifampicin and the trimethoprime-sulphamethoxazole combination.

The product according to the invention is particularly used in the treatment of the pathologies involving a strain of bacteria having a resistance, particularly nosocomial and/or community-aquired infections, such as abdominal infections, digestive infections, urinary infections, respiratory infections, neuro-meningeal infections, oro-pharyngeal infections, genital infections, endocarditis, infections of the skin and of the soft tissues, osteo-articular infections, ocular infections, septicaemia or bacteraemia, more particularly in the treatment of the pathologies listed below in Table 1.

TABLE 1

| | |
|---|---|
| abdominal infections | peritonitis, appendicitis etc. |
| digestive infections | collective food-poisoning diarrhoea, post- |
| | antibiotherapy diarrhoea etc. |
| urinary infections | cystitis, pyelonephritis, prostatitis etc. |
| respiratory infections | bronchitis, pneumonias, pneumopathies, |
| | abscess etc. |
| neuro-meningeal | Bacterial meningitis, cerebral abscess etc. |
| infections | |
| infections of the | sinusitis, otitis, anginas, phlegmons, |
| oro-pharyngeal sphere | epiglottiditis etc. |
| genital infections | vulvitis, vaginitis/vaginosis, cervicitis, |
| | salpingitis etc. |
| infections of the skin | furonculosis, abscess, eschar, diabetes foot etc. |
| and soft tissues | |
| ocular infections | conjunctivitis, keratitis, endophtalmias etc. |
| other infections | septicaemia or bacteraemia etc. |

In a particularly advantageous embodiment, the invention relates to a product comprising:
a calixarene represented by Formula II, and
a given antibiotic chosen from tigecycline, fusidic acid or fosfomycin for its use in the treatment of pathologies involving the MRSA strain without associated resistance.

In another particularly advantageous embodiment, the invention relates to a product comprising:
a calixarene represented by Formula II, and
a given antibiotic chosen from penicillin G, tigecycline, fusidic acid or fosfomycin for its use in the treatment of pathologies involving the MRSA strain having a resistance to the aminoglycosides and fluoroquinolones.

In another particularly advantageous embodiment, the invention relates to a product comprising:
a calixarene represented by Formula II, and
a given antibiotic chosen from penicillin G, imipenem, gentamicin, tigecycline, fusidic acid or fosfomycin for its use in the treatment of pathologies involving the MRSA strain having a resistance to the macrolides, fluoroquinolones, macrolides-lincosamides-synergistins and ofloxacin.

In another particularly advantageous embodiment, the invention relates to a product comprising:
a calixarene represented by Formula II, and
a given antibiotic chosen from tigecycline, fusidic acid or fosfomycin for its use in the treatment of pathologies involving the wild-type strain of Staphylococcus aureus.

By “a wild-type strain of Staphylococcus aureus” is meant a Staphylococcus aureus strain which has no mechanisms of acquired resistance to antibiotics (only natural resistances).

In another particularly advantageous embodiment, the invention relates to a product comprising:
a calixarene represented by Formula II, and
a given antibiotic chosen from cefotaxime, gentamicin or rifampicin for its use in the treatment of pathologies involving the ESBL-producing strain of Escherichia coli having an associated resistance to the aminoglycosides, rifampicin and the trimethoprime-sulphamethoxazole combination.

In another particularly advantageous embodiment, the invention relates to a product comprising:
a calixarene represented by Formula II, and
a given antibiotic chosen from gentamicin or rifampicin for its use in the treatment of pathologies involving the penicillinase-producing strain of Escherichia coli without associated resistance, or the cephalosporinase-hyperproducing strain of Escherichia coli having an associated resistance to the aminoglycosides, quinolones and the trimethoprime-sulfamethoxazole combination.

In another particularly advantageous embodiment, the invention relates to a product comprising:
a calixarene represented by Formula II, and
a given antibiotic chosen from gentamicin, tobramycin, or rifampicin for its use in the treatment of pathologies involving a wild-type strain of Escherichia coli.

By “a wild-type strain of Escherichia coli” is meant an E. coli strain which has no mechanisms of acquired resistance to antibiotics (only natural resistances).

In another particularly advantageous embodiment, the invention relates to a product comprising:
a calixarene represented by Formula II, and
a given antibiotic chosen from piperacillin-tazobactam, rifampicin, tobramycin, for its use in the treatment of pathologies involving a Pseudomonas aeruginosa strain, having a resistance to the β-lactams, the trimethoprime-sulphamethoxazole combination and fosfomycin.

In another particularly advantageous embodiment, the invention relates to a product comprising:
a calixarene represented by Formula II, and
a given antibiotic chosen from ceftazidime, rifampicin, colimycin, fosfomycin, for its use in the treatment of pathologies involving a strain of Pseudomonas aeruginosa having a resistance to the β-lactams (including the carbapenems), aminoglycosides, the trimethoprime-sulphamethoxazole combination and ciprofloxacin.

In another particularly advantageous embodiment, the invention relates to a product comprising:
a calixarene represented by Formula II, and
a given antibiotic chosen from piperacillin-tazobactam, imipenem, rifampicin, colimycin, fosfomycin, tobramycin and ciprofloxacin for its use in the treatment of pathologies involving a mucoid strain of Pseudomonas aeruginosa having a resistance to rifampicin and the trimethoprime-sulphamethoxazole combination.

In another particularly advantageous embodiment, the invention relates to a product comprising:
a calixarene represented by Formula II, and
a given antibiotic chosen from piperacillin-tazobactam, ceftazidime, tobramycin, ciprofloxacin, rifampicin, fosfomycin or ticarcilline-clavulanic acid for its use in the treatment of pathologies involving a wild-type strain of Pseudomonas aeruginosa.

By “a wild-type strain of Pseudomonas aeruginosa” is meant a Pseudomonas aeruginosa strain which has no mechanisms of acquired resistance to antibiotics (only natural resistances).

The present invention also relates to a pharmaceutical composition comprising at least one product as described above as an active substance in combination with a pharmaceutically acceptable vehicle.

Various formulations are possible for said pharmaceutical compositions: in the form of a gelatin capsule, tablet, powder, cream, lotion, aqueous or hydroalcoholic solution, mouthwash, eye drops, milk, foam, gel, spray or powder for example.

Said pharmaceutical composition can be administered by oral, parenteral, or topical route.

In such a pharmaceutical composition according to the invention, a person skilled in the art knows that the unit dose for administration of Cx1 depend of the nature of the bacteria to be treated, but also on the unit dose of a given antibiotic.

The unit dose for administration of a given standard antibiotic is known to a person skilled in the art.

The subject of another aspect of the present invention is to provide a combination product for simultaneous or separate use or spread over time for the treatment of pathologies involving at least one bacterial strain having a resistance.

Said combination product contains:
a given antibiotic chosen from: the β-lactams, the aminoglycosides, fluoroquinolones, fosfomycin, colimycin, rifampicin, tigecycline, or fusidic acid, and
a calixarene represented by Formula I below:

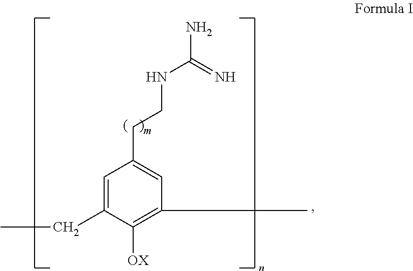

in which:

(i) n=an integer from 4 to 16,

(ii) m=an integer from 1 to 10,

(iii) X is chosen from:
a hydrogen,
an alkyl group, the number of carbons being from 1 to 20, in particular from 1 to 10,
a halogen chosen from Cl, Br, I, or
an amphiphilic group chosen from an anionic group, such as the carboxylates —RCO₂—, the sulphates —RSO₄—, the sulphonates —RSO₃—, a cationic group, such as RNH₃′, in which R is an alkyl group, the number of carbons being from 1 to 20, in particular from 1 to 10,
   as a combination product, for its simultaneous or separate use or spread over time for the treatment of pathologies involving at least one bacterial strain having an acquired resistance to at least one defined antibiotic, such as abdominal infections, digestive infections, urinary infections, respiratory infections, neuro-meningeal infections, oro-pharyngeal infections, genital infections, endocarditis, infections of the skin and of the soft tissues, osteo-articular infections, ocular infections, septicaemias or bacteraemias, more particularly the pathologies listed above in Table 1.

In such a combination product according to the invention, the calixarene represented by Formula I and the given antibiotic are present physically separated in an end product. The calixarene and the given antibiotic can be administered to patients simultaneously, separately or according to an order spread over time, according to the prescription.

In a particular embodiment, the invention relates to a combination product for its use as described above, in which the calixarene corresponds to the calixarene represented by Formula II below:

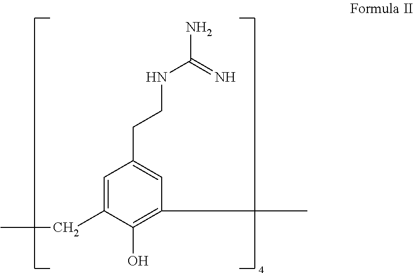

The molecule represented by Formula II is para-guanidinoethylcalix[4]rene, designated Cx1 in the present application.

In another advantageous embodiment of the combination product of the invention for its use as described above, the given antibiotic is chosen from imipenem, piperacillin-tazobactam, penicillin G, cefotaxime, ceftazidime, tobramycin, gentamicin, ciprofloxacin, rifampicin, fosfomycin, colimycin, streptomycin, ticarcilline-clavulanic acid, tigecycline or fusidic acid.

In another particular embodiment, the invention relates to a combination product for simultaneous or separate use or spread over time for the treatment of pathologies involving at least one resistant bacterial strain belonging to a species chosen from Escherichia coli, Pseudomonas aeruginosa, and Staphylococcus aureus.

In another particular embodiment, the combination product according to the invention is intended for simultaneous or separate use or spread over time for the treatment of pathologies involving at least one resistant bacterial strain chosen from:
a wild-type strain of Staphylococcus aureus
a Methicillin-Resistant Staphylococcus aureus strain (MRSA) without associated resistance,
an MRSA strain having a resistance to the aminoglycosides and fluoroquinolones,
an MRSA strain having a resistance to the aminoglycosides, fluoroquinolones, macrolides-lincosamides-synergystins and ofloxacin,
a wild-type strain of Escherichia coli
a penicillinase-producing strain of Escherichia coli without associated resistance,
an ESBL (Extended-Spectrum β-Lactamase)-producing strain of Escherichia coli, having associated resistance to the aminoglycosides, rifampicine and the trimethoprime-sulphamethoxazole combination,
a cephalosporinase-hyperproducing strain of Escherichia coli having an associated resistance to the aminoglycosides, quinolones and the trimethoprime-sulphamethoxazole combination,
a wild-type strain of Pseudomonas aeruginosa
a Pseudomonas aeruginosa strain having a resistance to the β-lactams, the trimethoprime-sulphamethoxazole combination and to fosfomycine,
a Pseudomonas aeruginosa strain having a resistance to the β-lactams (including the carbapenems), aminoglycosides, the trimethoprime-sulphamethoxazole combination and ciprofloxacin,
a mucoid strain of Pseudomonas aeruginosa having a resistance to rifampicin and the trimethoprime-sulphamethoxazole combination.

In a particular embodiment, the invention relates to a product containing the calixarene of Formula II (Cx1) and a given antibiotic chosen from tigecycline, fusidic acid or fosfomycin, as a combination product, for simultaneous or separate use or spread over time for the treatment of pathologies involving the MRSA strain without associated resistance.

In another particular embodiment, the invention relates to a product containing the calixarene of Formula II (Cx1) and a given antibiotic chosen from penicillin G, tigencyline, fusidic acid or the fosfomycin, as a combination product for simultaneous or separate use or spread over time for the treatment of pathologies involving the MRSA strain having a resistance to the aminoglycosides and fluoroquinolones.

In another particular embodiment, the invention relates to a product containing the calixarene of Formula II (Cx1) and a given antibiotic chosen from penicilline G, imipenem, gentamicin, tigecycline, fusidic acid or fosfomycin, as a combination product for simultaneous or separate use or spread over time for the treatment of pathologies involving the MRSA strain having a resistance to the aminoglycosides, fluoroquinolones, macrolides-lincosamides-synergistins and ofloxacine.

In another particular embodiment, the invention relates to a product containing the calixarene of Formula II (Cx1) and a given antibiotic chosen from tigecycline, fusidic acid or fosfomycin, as a combination product for simultaneous or separate use or spread over time for the treatment of pathologies involving the wild-type strain of Staphylococcus aureus.

In another particular embodiment, the invention relates to a product containing the calixarene of Formula II (Cx1) and a given antibiotic chosen from cefotaxim, gentamicin or rifampicin, as a combination product for simultaneous or separate use or spread over time for the treatment of pathologies involving the ESBL-producing strain of Escherichia coli having an associated resistance to the aminoglycosides, rifampicin and the trimethoprime-sulphamethoxazole combination.

In another particular embodiment, the invention relates to a product containing the calixarene of Formula II (Cx1) and a given antibiotic chosen from gentamicin or rifampicin, as a combination product for simultaneous or separate use or spread over time for the treatment of pathologies involving the penicillinase-producing strain of Escherichia coli without associated resistance, or the cephalosporinase-hyperproducing strain of Escherichia coli having an associated resistance to the aminoglycosides, quinolones and the trimethoprime-sulphamethoxazole combination.

In another particular embodiment, the invention relates to a product containing the calixarene of Formula II (Cx1) and a given antibiotic chosen from gentamicin, tobramycin, or rifampicin, as a combination product, for its simultaneous or separate use or spread over time for the treatment of pathologies involving the wild-type strain of Escherichia coli.

In another particular embodiment, the invention relates to a product containing the calixarene of Formula II (Cx1) and a given antibiotic chosen from piperacilline-tazobactam, rifampicin, tobramycin, as a combination product, for its simultaneous or separate use or spread over time for the treatment of pathologies involving a Pseudomonas aeruginosa strain having a resistance to the β-lactams, the trimethoprime-sulphamethoxazole combination and fosfomycin.

In another particular embodiment, the invention relates to a product containing the calixarene of Formula II (Cx1) and a given antibiotic chosen from ceftazidime, rifampicin, colimycin, fosfomycin for simultaneous or separate use or spread over time for the treatment of pathologies involving a Pseudomonas aeruginosa strain having a resistance to the β-lactams (including the carbapenems), aminoglycosides, the trimethoprime-sulphamethoxazole combination and ciprofloxacin.

In another particular embodiment, the invention relates to a product containing the calixarene of Formula II (Cx1) and a given antibiotic chosen from piperacillin-tazobactam, imipenem, rifampicin, colimycin, fosfomycin, tobramycin and ciprofloxacin, as a combination product, for its simultaneous or separate use or spread over time for the treatment of pathologies involving a mucoid strain of Pseudomonas aeruginosa having a resistance to rifampicin and the trimethoprime-sulphamethoxazole combination.

In another particular embodiment, the invention relates to a product containing the calixarene of Formula II (Cx1) and a given antibiotic chosen from piperacillin-tazobactam, ceftazidime, tobramycin, ciprofloxacin, rifampicin, fosfomycin or ticarcillin-clavulanic acid, as a combination product, for its simultaneous or separate use or spread over time for the treatment of pathologies involving a wild-type strain of Pseudomonas aeruginosa.

The illustrative figures and the examples given below by way of example can in no way be interpreted as limiting the scope of the invention.

### FIGURES

FIG. 1: FIG. 1 represents a microplate prepared for measuring the susceptibility of a bacterial strain to a solution containing an antibiotic and Cx1 respectively in a proportion of defined concentrations. Columns 1 and 12 contain only the control medium. Columns 2 and 11 contain the control medium and bacteria, but no antibiotic or Cx1. Columns 3 to 10 contain the bacteria, Cx1 in decreasing concentration and an antibiotic in increasing concentration.

FIG. 2A: FIG. 2A shows an additivity between Cx1 and another antibiotic.

FIG. 2B: FIG. 2B shows an indifference between Cx1 and another antibiotic.

FIG. 2C: FIG. 2C shows a synergism between Cx1 and another antibiotic.

FIG. 2D: FIG. 2D shows an antagonism between Cx1 and another antibiotic.

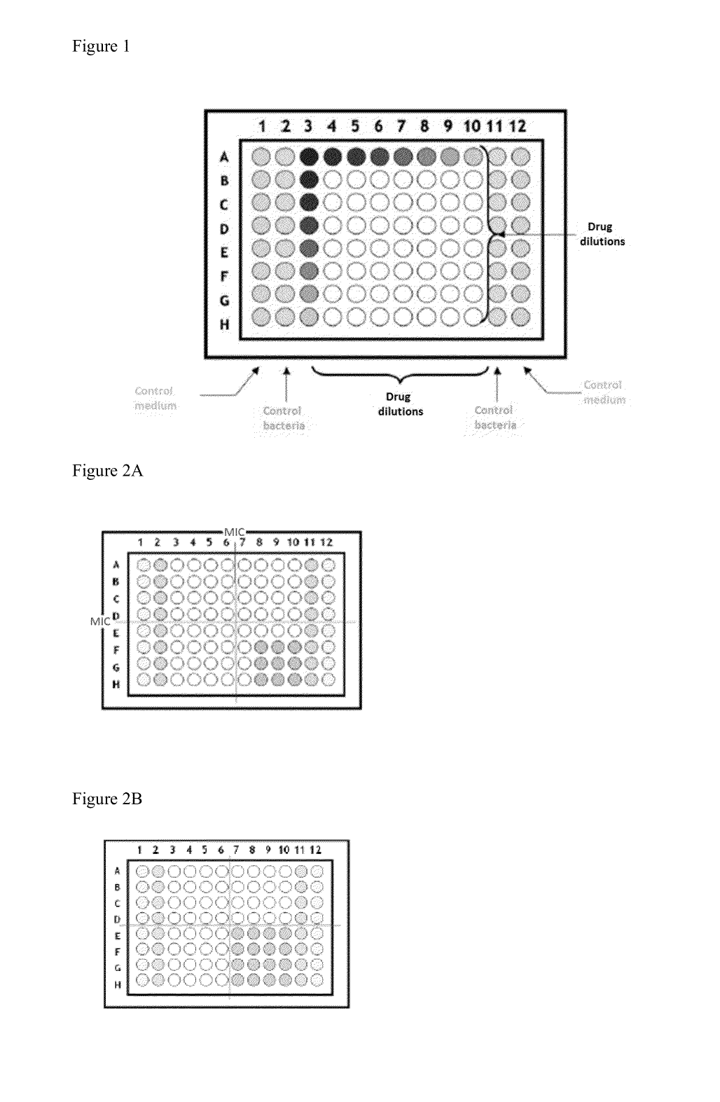

### RESULTS

### 1. Equipment and Method

1.1 Equipment and Reagent
10, 20 or 50 mL syringe
0.22 μm filter (Millex®GP, 0.22 μm filters, Millipore, France)
Falcon 15 and 50 mL tubes
96-well plates (Greiner, 650161)
Mueller Hinton Agars (MHA) (Difco, 225250)
Mueller Hinton Broths (MHB) (Difco, 275730)
Sterile distilled water

Solution of the drug to be tested: Cx1 (M=1221.11 g/mol) supplied by Prof. Regnouf de Vains in the form of white powder, taken up in sterile distilled water and filtered through a 0.22 μm filter to obtain a 10-2 mol/L sterile solution. The antibiotic was obtained commercially from the manufacturers, in the form of a ready-to-use sterile powder.

1.2 Bacterial Strains

Three reference strains were used, corresponding to those studied for the MICs and MBCs (Minimum Bactericidal Concentration): Escherichia coli ATCC 25922, Staphylococcus aureus ATCC 29213, Pseudomonas aeruginosa ATCC 27853. For each of these strains 3 corresponding clinical isolates were chosen, having various antibiotic-resistance profiles, routinely used in standard fashion:
EcR1, EcR2, EcR3;
SaR1, SaR3, SaR4;
PaR2, PaR3, PaR5.
   The antibiotic susceptibility profiles of these clinical isolates are shown in Annex 1.
   The fluctuations relative to the associated resistance type for certain strains are due to the development of the French (CA-SFM), European (EUCAST) and American (CLSI) recommendations for the categorization of the bacterial strains.

1.3. Procedure: Chessboard Technique

D-1: Culturing the Bacteria on MHA (Mueller Hinton Agar)

Incubation for 24 h at 35° C.

D0: Seeding an MHB (Mueller Hinton Broth)

Take an “average” colony from the agar D-1 and seed 5 mL of MHB.

Incubation for 24 h at 35° C.

D1: Preparation of the 96-Well Plates

Preparation of the Bacterial Inoculum:

The purity of the strains is verified by the absence of contaminants on the MHA seeded in parallel with the broth, and by carrying out Gram staining.

The bacterial suspension is transferred to a 15 mL Falcon tube, centrifuged for 10 min at 4500 g, then the pellet is re-suspended in 1 mL of sterile distilled water. Suitable dilutions are then prepared in order to obtain a bacterial inoculum between 5.10⁵ and 5.10⁶ CFU/mL.

Preparation of the Solutions: 1) Antibiotic (ATB) to be Tested, 2) Cx1

The MICs of the antibiotics were previously defined for each strain, by the method of microdilution in a liquid medium (CLSI (Clinical and Laboratory Standards Institute), 2003).

Suitable dilutions are prepared in order to obtain a solution having a concentration equivalent to 32 times the MIC of the ATB to be tested, in an MH (Mueller-Hinton) medium. Then a series of two-fold dilutions was prepared in MH medium in order to obtain the following concentrations: 16, 8, 4, 2, 1, 0.5 and 0.25 times the MIC (15 mL Falcon tubes). The same procedure is followed for the Cx1. This makes it possible to obtain a concentration range from 8 to 0.06 times the MIC in the microplate for the two molecules (dilution by half with addition of the 2nd molecule, then new dilution by ½ after addition of the bacterial suspension). Thus 64 ATB/Cx1 combinations are obtained.

For each plate, 1 mL of solution of each dilution is necessary.

Preparation of the Microplates

The final volume contained in each of the wells must be 100 μL. Two controls must be present on each plate:
column 1 and 12: medium control
column 2 and 11: medium+bacteria control

Distribution of the MH Medium

100 μL in each of the wells of columns 1 and 12.

50 μL in each of the wells of columns 2 and 11.

Distribution of the Dilution Range of the Antibiotic to be Tested and of the Molecule of Interest

Cx1: 25 μL in the wells of columns 11 to 3, starting with the lowest concentration.

ATB: 25 μL in the wells of rows H to A (columns 3 to 11), starting with the lowest concentration.

Distribution of the Bacterial Suspension

50 μL in each of the wells of columns 2 to 11.

D2: Reading the Turbidity at 540 Nm.

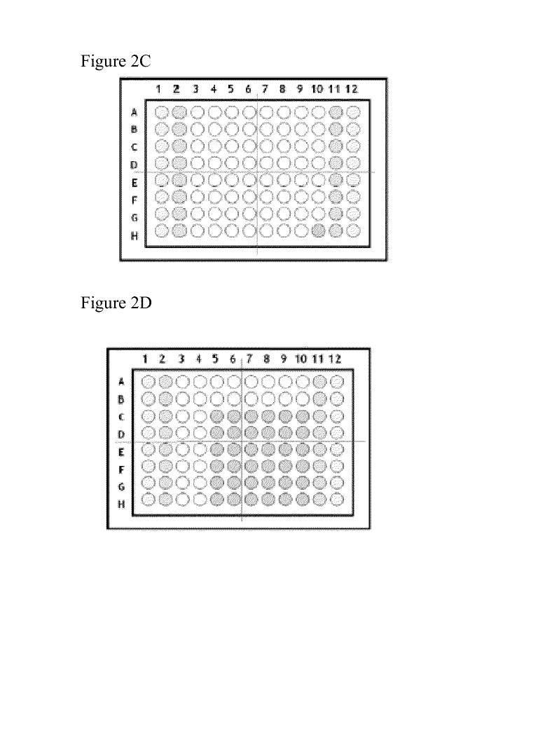

The different types of interactions observed by the chessboard technique are shown in FIGS. 2A, 2B, 2C and 2D:

The FIC (Fractional Inhibitory Concentration Index) value is defined by the following formula:

$$FIC = {FIC}_{A} + {FIC}_{B} = \frac{{MIC}_{A} in comb .}{{MIC}_{A} alone} + \frac{{MIC}_{B} in comb .}{{MIC}_{B} alone}$$

MIC_{A} in combination MIC_{A} alone MIC_{B} in combination MIC_{B} alone

When FIC≦0.5, there is a synergistic effect between antibiotic A and antibiotic B.

When 0.5<FIC≦1, there is an additive effect between antibiotic A and antibiotic B.

When 1<FIC≦4, there is an indifferent effect between antibiotic A and antibiotic B.

When FIC>4, there is an antagonistic effect between antibiotic A and antibiotic B.

For each strain and each antibiotic/Cx1 combination, the experiments were repeated a minimum of 3 times.

### 2. Results & Discussion

The results obtained are presented in the form of tables (Tables I to XII), showing in greater detail and by ATB/Cx1 pair:
the FIC indices obtained during the different experiments;
the optimum concentrations for the synergism;
the MICs of the compounds used alone;
the ranges tested and the nature of the interaction observed.

The results below show that no antagonism was observed between Cx1 and an antibiotic tested, irrespective of the strains and combinations tested.

The results also demonstrate that the treatment with Cx1 in combination with the treatment with an antibiotic for a pathology involving a bacterial strain resistant to said antibiotic makes it possible:
to confer upon the bacterial strain a certain level of susceptibility to said antibiotic;
to reduce the dose of said antibiotic as well as the dose of Cx1 administered.

The results demonstrate that the treatment with Cx1 in combination with the treatment with a antibiotic for a pathology involving a bacterial strain having a resistance to a defined antibiotic makes it possible to reduce the dose of antibiotic as well as the dose of Cx1 administered.

These results are equally valid for the treatment with Cx1 in combination with the treatment with an antibiotic for a pathology involving a bacterial strain having a resistance to at least two different families of antibiotics. This is the case with the tested strains SaR3, EcR2, SaR4, PaR2 and PaR3.

Among all of the strains analyzed, Pseudomonas aeruginosa is the strain for which the greatest number of synergistic combinations with very varied antibiotics (β-lactams, aminoglycosides, fluoroquinolones, etc.), was observed.

Moreover, the antibiotics for which a synergism between same and Cx1 was observed, act at the level of:
the wall: fosfomycin (in the knowledge that the specific mechanism of action of fosfomycin is demonstrated by quasi-constant synergistic activity with the other antibiotics active on the bacterial wall) but also with the piperacillin-tazobactam & ticarcillin-clavulanic acid combinations, and ceftazidime;
protein synthesis: tigecycline, gentamicin, tobramycin, streptomycin, fusidic acid;
nucleic acid synthesis: rifampicin, ciprofloxacin.
   2. Synergism of the Combination of Cx1 with Antibiotics Against Escherichia coli Strains, Antibiotic-Resistant or not

TABLE I
Synergism against the Escherichia coli strain ATCC 25922 (wild type) (n = 4)

| | Initial | Ranges | | | | | |
|---|---|---|---|---|---|---|---|
| | MICs | tested | FIC | Optimum MICs | Difference | Difference | |
| Combinations | (mg/L) | (mg/L) | index | (mg/L) | Cx1 MIC | ATB MIC | Finding |
| Cx1/Amoxicillin | 4/4 | 32/32 | 1-4 | nd | nd | Nd | Indifference |
| Cx1/Amoxicillin- | 4/4 | 16/256 | 0.53-1 | 0.125/2 | ↓ x5 | ↓ x1 | Additivity |
| clavulanic acid | | | | | | | |
| Cx1/Piperacillin | 4/2 | 16/256 | 1-4 | nd | nd | nd | Indifference |
| Cx1/Cefotaxime | 4/0.06 | 16/1 | 0.53-1 | 0.125/0.03 | ↓ x5 | ↓ x1 | Additivity |
| Cx1/Ceftazidime | 4/0.125 | 16/1 | 0.62-0.98 | 2/0.015 | ↓ x1 | ↓ x3 | Additivity |
| Cx1/Imipenem | 4/0.125 | 16/1 | 1-4 | nd | nd | nd | Indifference |
| Cx1/Ertapenem | 4/0.015 | 16/1 | 0.625-1 | 1/0.07 | ↓ x2 | ↓ x1 | Additivity |
| Cx1/Gentamicin | 4/0.125 | 16/4 | 0.27-0.98 | 0.125/0.03 | ↓ x5 | ↓ x2 | Synergism |
| Cx1/Amikacin | 4/0.25 | 16/4 | 1-4 | nd | nd | nd | Indifference |
| Cx1/Tobramycin | 4/0.25 | 16/4 | 0.365-1 | 0.5/0.06 | ↓ x3 | ↓ x2 | Synergism |
| Cx1/Ciprofloxacin | 2/0.015 | 16/1 | 1-4 | nd | nd | nd | Indifference |
| Cx1/Rifampicin | 4/4 | 16/16 | 0.16-1 | 0.5/0.125 | ↓ x3 | ↓ x5 | Synergism |

TABLE II
Synergism against a penicillinase-producing strain of Escherichia coli without
associated resistance (or EcR1) (n = 4)

| | Initial | Ranges | | Optimum | | | |
|---|---|---|---|---|---|---|---|
| | MICs | tested | FIC | concentrations | Difference | Difference | |
| Combinations | (mg/L) | (mg/L) | index | (mg/L) | Cx1 MIC | ATB MIC | Finding |
| Cx1/Amoxicillin | 2/>256 | 32/256 | 1-4 | nd | nd | nd | Indifference |
| Cx1/Amoxicillin- | 2/16 | 16/256 | 1-4 | nd | nd | nd | Indifference |
| clavulanic acid | | | | | | | |
| Cx1/Piperacillin | 2/256 | 16/256 | 1-4 | nd | nd | nd | Indifference |
| Cx1/Cefotaxime | 2/0.06 | 16/1 | 0.75-1 | 1/0.015 | ↓ x1 | ↓ x2 | Additivity |
| Cx1/Ceftazidime | 2/0.25 | 16/1 | 0.56-1 | 0.125/0.125 | ↓ x4 | ↓ x1 | Additivity |
| Cx1/Imipenem | 2/0.125 | 16/1 | 1-4 | nd | nd | nd | Indifference |
| Cx1/Ertapenem | 2/0.015 | 16/1 | 0.75-1 | 0.5/0.07 | ↓ x2 | ↓ x1 | Additivity |
| Cx1/Gentamicin | 2/0.125 | 16/4 | 0.3-1 | 0.125/0.03 | ↓ x4 | ↓ x2 | Synergism |
| Cx1/Amikacin | 2/0.25 | 16/4 | 1-4 | nd | nd | nd | Indifference |
| Cx1/Tobramycin | 2/0.25 | 16/4 | 1-4 | nd | nd | nd | Indifference |
| Cx1/Ciprofloxacin | 2/0.015 | 16/1 | 1-4 | nd | nd | nd | Indifference |
| Cx1/Rifampicin | 2/4 | 16/16 | 0.28-1 | 0.5/0.125 | ↓ x2 | ↓ x5 | Synergism |

TABLE III
Synergism against an ESBL-producing strain of Escherichia coli, having an
associated resistance to the aminoglycosides (or EcR3) (n = 4)

| | Initial | Ranges | | Optimum | | | |
|---|---|---|---|---|---|---|---|
| | MICs | tested | | concentrations | Difference | Difference | |
| Combinations | (mg/L) | (mg/L) | FIC index | (mg/L) | Cx1 MIC | ATB MIC | Finding |
| Cx1/Amoxicillin | 2/>256 | 16/256 | 1-4 | nd | nd | nd | Indifference |
| Cx1/Amoxicillin- | 2/32 | 16/256 | 0.56-1 | 0.125/16 | ↓ x4 | ↓ x1 | Additivity |
| clavulanic acid | | | | | | | |
| Cx1/Piperacillin | 2/128 | 16/256 | 1-4 | nd | nd | nd | Indifference |
| Cx1/Cefotaxime | 2/128 | 16/256 | 0.375-1 | 0.25/32 | ↓ x3 | ↓ x2 | Synergism |
| Cx1/Ceftazidime | 2/1 | 16/16 | 0.56-1 | 0.125/0.5 | ↓ x4 | ↓ x1 | Additivity |
| Cx1/Imipenem | 2/0.125 | 16/1 | 1-4 | nd | nd | nd | Indifference |
| Cx1/Ertapenem | 2/0.0015 | 16/1 | 1-4 | nd | nd | nd | Indifference |
| Cx1/Gentamicin | 2/2 | 16/4 | 0.31-1 | 0.125/0.5 | ↓ x4 | ↓ x3 | Synergism |
| Cx1/Amikacin | 2/1 | 16/4 | 1-4 | nd | nd | nd | Indifference |
| Cx1/Tobramycin | 2/4 | 16/4 | 1-4 | nd | nd | nd | Indifference |
| Cx1/Ciprofloxacin | 2/0.06 | 16/1 | 0.56-1 | 0.125/0.03 | ↓ x4 | ↓ x1 | Additivity |
| Cx1/Rifampicin | 2/4 | 16/16 | 0.25-1 | 0.25/0.5 | ↓ x3 | ↓ x3 | Synergism |

TABLE IV
Synergism against a cephalosporinase-hyperproducing strain of Escherichia coli having
an associated resistance to the aminoglycosides, quinolones and the trimethoprim-
sulphamethoxazole combination (or EcR2) (n = 4)

| | Initial | | | Optimum | | | |
|---|---|---|---|---|---|---|---|
| | MICs | Ranges tested | FIC | concentrations | Difference | Difference | |
| Combinations | (mg/L) | (mg/L) | index | (mg/L) | Cx1 MIC | ATB MIC | Finding |
| Cx1/Amoxicillin | 2/256 | 16/256 | 1-4 | nd | nd | nd | Indifference |
| Cx1/Amoxicillin- | 2/256 | 32/256 | 0.56-1 | 0.125/128 | ↓ x4 | ↓ x1 | Additivity |
| clavulanic acid | | | | | | | |
| Cx1/Piperacillin | 2/32 | 16/256 | 0.56-1 | 0.125/16 | ↓ x4 | ↓ x1 | Additivity |
| Cx1/Cefotaxime | 2/4 | 16/32 | 1-4 | nd | nd | nd | Indifference |
| Cx1/Ceftazidime | 2/8 | 16/32 | 1-4 | nd | nd | nd | Indifference |
| Cx1/Imipenem | 2/0.125 | 16/1 | 1-4 | nd | nd | nd | Indifference |
| Cx1/Ertapenem | 2/0.03 | 16/1 | 1-4 | nd | nd | nd | Indifference |
| Cx1/Gentamicin | 2/2 | 16/4 | 0.31-1 | 0.125/0.5 | ↓ x4 | ↓ x2 | Synergism |
| Cx1/Amikacin | 2/0.5 | 16/4 | 1-4 | nd | nd | nd | Indifference |
| Cx1/Tobramycin | 2/4 | 16/4 | 1-4 | nd | nd | nd | Indifference |
| Cx1/Ciprofloxacin | 2/0.015 | 16/1 | 1-4 | nd | nd | nd | Indifference |
| Cx1/Rifampicin | 2/2 | 16/16 | 0.31-1 | 0.25/0.5 | ↓ x3 | ↓ x2 | Synergism |

3. Synergism of the Combination of Cx1 with Antibiotics Against Staphylococcus aureus Strains, Antibiotic-Resistant or not

TABLE V
Synergism against the Staphylococcus aureus strain ATCC 29213 (wild type) (n = 4)

| | Initial | Ranges | | Optimum | | | |
|---|---|---|---|---|---|---|---|
| | MICs | tested | FIC | concentrations | Difference | Difference | |
| Combinations | (mg/L) | (mg/L) | index | (mg/L) | Cx1 MIC | ATB MIC | Finding |
| Cx1/Penicillin G | 8/1 | 64/4 | 0.5-1 | 2/0.25 | ↓ x2 | ↓ x2 | Additivity |
| Cx1/Imipenem | 8/0.015 | 64/1 | 1-4 | nd | nd | nd | Indifference |
| Cx1/Erythromycin | 8/0.5 | 64/4 | 1-4 | nd | nd | nd | Indifference |
| Cx1/Vancomycin | 8/0.5 | 64/4 | 0.75-1 | 4/0.125 | ↓ x1 | ↓ x2 | Additivity |
| Cx1/Levofloxacin | 8/0.125 | 64/4 | 1-4 | nd | nd | nd | Indifference |
| Cx1/Amikacin | 8/1 | 64/4 | 0.53-1 | 4/0.03 | ↓ x1 | ↓ x5 | Additivity |
| Cx1/Gentamicin | 8/0.25 | 64/2 | 0.49-1 | 2/0.06 | ↓ x2 | ↓ x2 | Additivity |
| Cx1/Streptomycin | 8/4 | 64/32 | 0.375-1 | 1/1 | ↓ x3 | ↓ x2 | Synergism |
| Cx1/Linezolid | 8/2 | 64/8 | 1-4 | nd | nd | nd | Indifference |
| Cx1/Tigecycline | 8/0.25 | 64/2 | 0.18-1 | 0.5/0.03 | ↓ x4 | ↓ x3 | Synergism |
| Cx1/Fusidic acid | 8/2 | 64/8 | 0.125-1 | 0.5/0.125 | ↓ x4 | ↓ x4 | Synergism |
| Cx1/Fosfomycin | 8/8 | 64/32 | 0.18-1 | 0.5/1 | ↓ x4 | ↓ x3 | Synergism |

TABLE VI
Synergism against an MRSA strain without associated resistance (or SaR1) (n = 4)

| | | Ranges | | Optimum | | | |
|---|---|---|---|---|---|---|---|
| | MIC | tested | FIC | concentrations | Difference | Difference | |
| Combinations | (mg/L) | (mg/L) | index | (mg/L) | Cx1 MIC | ATB MIC | Finding |
| Cx1/Penicillin G | 8/0.5 | 64/4 | 0.5-1 | 2/0.125 | ↓ x2 | ↓ x2 | Additivity |
| Cx1/Imipenem | 8/0.25 | 64/4 | 0.625-1 | 0.5/0.06 | ↓ x4 | ↓ x2 | Additivity |
| Cx1/Erythromycin | 8/0.5 | 64/4 | 1-4 | nd | nd | nd | Indifference |
| Cx1/Vancomycin | 8/0.25 | 64/4 | 1-4 | nd | nd | nd | Indifference |
| Cx1/Levofloxacin | 8/0.25 | 64/4 | 1-4 | nd | nd | nd | Indifference |
| Cx1/Gentamicin | 8/0.25 | 64/2 | 0.49-1 | 2/0.06 | ↓ x2 | ↓ x2 | Additivity |
| Cx1/Streptomycin | 8/4 | 64/32 | 0.75-1 | 4/1 | ↓ x1 | ↓ x2 | Additivity |
| Cx1/Linezolid | 8/2 | 64/8 | 1-4 | nd | nd | nd | Indifference |
| Cx1/Tigecycline | 8/0.25 | 64/2 | 0.18-1 | 0.5/0.03 | ↓ x4 | ↓ x3 | Synergism |
| Cx1/Fusidic acid | 8/0.5 | 64/8 | 0.245-1 ’ | 1/0.06 | ↓ x3 | ↓ x3 | Synergism |
| Cx1/Fosfomycin | 8/2 | 64/32 | 0.31-1 | 0.5/0.5 | ↓ x4 | ↓ x2 | Synergism |

TABLE VII
Synergism against an MRSA strain having a resistance to the aminoglycosides and
fluoroquinolones (SaR3) (n = 4)

| | | Ranges | | Optimum | | | |
|---|---|---|---|---|---|---|---|
| | MIC | tested | | concentrations | Difference | Difference | |
| Combinations | (mg/L) | (mg/L) | FIC index | (mg/L) | Cx1 MIC | ATB MIC | Finding |
| Cx1/Penicillin G | 8/0.5 | 64/4 | 0.375-1 | 2/0.06 | ↓ x2 | ↓ x3 | Synergism |
| Cx1/Imipenem | 8/0.25 | 64/4 | 0.56-1 | 2/0.06 | ↓ x2 | ↓ x2 | Additivity |
| Cx1/Erythromycin | 8/0.5 | 64/4 | 1-4 | nd | nd | nd | Indifference |
| Cx1/Vancomycin | 8/0.25 | 64/4 | 1-4 | nd | nd | nd | Indifference |
| Cx1/Levofloxacin | 8/8 | 64/32 | 1-4 | nd | nd | nd | Indifference |
| Cx1/Gentamicin | 8/0.125 | 64/2 | 0.74-1 | 4/0.03 | ↓ x1 | ↓ x2 | Additivity |
| Cx1/Streptomycin | 8/4 | 64/32 | 0.625-1 | 1/2 | ↓ x3 | ↓ x1 | Additivity |
| Cx1/Linezolid | 8/2 | 64/8 | 1-4 | nd | nd | nd | Indifference |
| Cx1/Tigecycline | 8/0.25 | 64/2 | 0.245-1 | 1/0.03 | ↓ x3 | ↓ x3 | Synergism |
| Cx1/Fusidic acid | 8/0.25 | 64/8 | 0.365-1 | 1/0.06 | ↓ x3 | ↓ x2 | Synergism |
| Cx1/Fosfomycin | 8/16 | 64/32 | 0.18-1 | 0.5/2 | ↓ x4 | ↓ x3 | Synergism |

TABLE VIII
Synergism against an MRSA strain having a resistance to the aminoglycosides,
fluoroquinolones, macrolides-lincosamides-synergistins and ofloxacin (SaR4) (n = 4)

| | | Ranges | | Optimum | | | |
|---|---|---|---|---|---|---|---|
| | MIC | tested | FIC | concentrations | Difference | Difference | |
| Combinations | (mg/L) | (mg/L) | index | (mg/L) | Cx1 MIC | ATB MIC | Finding |
| Cx1/Penicillin G | 8/4 | 64/32 | 0.375-1 | 2/0.5 | ↓ x2 | ↓ x3 | Synergism |
| Cx1/Imipenem | 8/2 | 64/32 | 0.375-1 | 1/0.5 | ↓ x3 | ↓ x2 | Synergism |
| | | | | 2/0.25 | ↓ x2 | ↓ x3 | |
| Cx1/Erythromycin | 8/>32 | 64/32 | 1-4 | nd | nd | nd | Indifference |
| Cx1/Vancomycin | 8/0.25 | 64/4 | 1-4 | nd | nd | nd | Indifference |
| Cx1/Levofloxacin | 8/>32 | 64/32 | 1-4 | nd | nd | nd | Indifference |
| Cx1/Gentamicin | 8/0.25 | 64/2 | 0.30-1 | 0.5/0.06 | ↓ x4 | ↓ x2 | Synergism |
| Cx1/Streptomycin | 8/4 | 64/32 | 0.625-1 | 4/0.5 | ↓ x1 | ↓ x3 | Additivity |
| Cx1/Linezolid | 8/2 | 64/8 | 0.56-1 | 0.5/1 | ↓ x4 | ↓ x1 | Additivity |
| Cx1/Tigecycline | 8/0.25 | 64/2 | 0.18-1 | 0.5/0.03 | ↓ x4 | ↓ x3 | Synergism |
| Cx1/Fusidic acid | 8/4 | 64/8 | 0.08-1 | 0.5/0.06 | ↓ x4 | ↓ x6 | Synergism |
| Cx1/Fosfomycin | 8/128 | 64/32 | 1-4 | nd | nd | nd | Indifference |

4. Synergism of the Combination of Cx1 with Antibiotics Against Pseudomonas aeruginosa Strains, Antibiotic-Resistant or not

TABLE IX
Synergism against the Pseudomonas aeruginosa strain ATCC 27853 (wild type) (n = 4)

| | | Ranges | | Optimum | | | |
|---|---|---|---|---|---|---|---|
| | MIC | tested | | concentrations | Difference | Difference | |
| Combinations | (mg/L) | (mg/L) | FIC index | (mg/L) | Cx1 MIC | ATB MIC | Finding |
| Cx1/Ticarcillin- | 32/8 | 256/32 | 0.375-1 | 4/1 | ↓ x3 | ↓ x3 | Synergism |
| clavulanic acid | | | | | | | |
| Cx1/Piperacillin- | 32/16 | 256/128 | 0.31-1 | 2/4 | ↓ x4 | ↓ x2 | Synergism |
| Tazobactam | | | | | | | |
| Cx1/Ceftazidime | 32/4 | 256/64 | 0.375-1 | 4/1 | ↓ x3 | ↓ x2 | Synergism |
| Cx1/Imipenem | 32/2 | 256/16 | 0.5-4 | 8/0.5 | ↓ x2 | ↓ x2 | Additivity |
| Cx1/Rifampicin | 32/64 | 256/256 | 0.12-1 | 2/4 | ↓ x4 | ↓ x4 | Synergism |
| Cx1/Colimycin | 32/4 | 256/64 | 0.5-1 | nd | nd | nd | Additivity |
| Cx1/Fosfomycin | 32/16 | 256/256 | 0.375-1 | 4/4 | ↓ x3 | ↓ x2 | Synergism |
| | | | | 2/8 | ↓ x4 | ↓ x1 | |
| Cx1/Tobramycin | 32/0.5 | 256/4 | 0.18-1 | 2/0.06 | ↓ x4 | ↓ x3 | Synergism |
| Cx1/Amikacin | 32/0.5 | 256/4 | 1-4 | nd | nd | nd | Indifference |
| Cx1/Ciprofloxacin | 32/0.5 | 256/4 | 0.185-1 | 4/0.125 | ↓ x3 | ↓ x2 | Synergism |

TABLE X
Synergism against a Pseudomonas aeruginosa strain having a resistance to β-lactams, the
trimethoprim-sulphamethoxazole combination and fosfomycin (or PaR2) (n = 4)

| | | | | Optimum | | | |
|---|---|---|---|---|---|---|---|
| | MIC | Ranges tested | | concentrations | Difference | Difference | |
| Combinations | (mg/L) | (mg/L) | FIC index | (mg/L) | Cx1 MIC | ATB MIC | Finding |
| Cx1/Ticarcillin-Clavulanic | 32/512 | 256/512 | 1-4 | nd | nd | nd | Indifference |
| acid | | | | | | | |
| Cx1/Piperacillin-Tazobactam | 32/512 | 256/512 | 0.25-1 | 4/64 | ↓x3 | ↓x3 | Synergism |
| Cx1/Ceftazidime | 32/8 | 256/64 | 1-4 | nd | nd | nd | Indifference |
| Cx1/Imipenem | 32/2 | 256/16 | 0.5-4 | 8/1 | ↓x2 | ↓x1 | Additivity |
| Cx1/Rifampicin | 32/64 | 256/256 | 0.185-1 | 4/4 | ↓x3 | ↓x4 | Synergism |
| | | | | 8/2 | ↓x2 | ↓x5 | |
| Cx1/Colimycin | 32/8 | 256/64 | 1-4 | nd | nd | nd | Indifference |
| Cx1/Fosfomycin | 32/64 | 256/256 | 0.5-4 | 16/32 | ↓x1 | ↓x1 | Additivity |
| Cx1/Tobramycin | 32/1 | 256/4 | 0.31-1 | 2/0.25 | ↓x4 | ↓x2 | Synergism |
| Cx1/Amikacin | 32/1 | 256/4 | 1-4 | nd | nd | nd | Indifference |
| Cx1/Ciprofloxacin | 32/1 | 256/4 | 0.5 | nd | nd | nd | Additivity |

TABLE XI
Synergism against a Pseudomonas aeruginosa strain having a resistance to β-lactams (including the carbapenems),
aminoglycosides, the trimethoprim-sulphamethoxazole combination and ciprofloxacin (or PaR3) (n = 4)

| | | | | Optimum | | | |
|---|---|---|---|---|---|---|---|
| | MIC | Ranges tested | | concentrations | Difference | Difference | |
| Combinations | (mg/L) | (mg/L) | FIC index | (mg/L) | Cx1 MIC | ATB MIC | Finding |
| Cx1/Ticarcillin-Clavulanic acid | 32/512 | 256/512 | 1-4 | nd | nd | nd | Indifference |
| Cx1/Piperacillin-Tazobactam | 32/512 | 256/512 | 1-4 | nd | nd | nd | Indifference |
| Cx1/Ceftazidime | 32/16 | 256/64 | 0.31-1 | 2/4 | ↓ x4 | ↓ x2 | Synergism |
| Cx1/Imipenem | 32/32 | 256/256 | 0.5-1 | 8/8 | ↓ x2 | ↓ x2 | Additivity |
| Cx1/Rifampicin | 32/32 | 256/256 | 0.09-1 | 2/2 | ↓ x4 | ↓ x4 | Synergism |
| Cx1/Colimycin | 32/8 | 256/64 | 0.155-1 | 4/0.5 | ↓ x3 | ↓ x4 | Synergism |
| Cx1/Fosfomycin | 32/>64 | 256/256 | 0.31-1 | 2/64 | ↓ x4 | >↓ x2 | Synergism |
| Cx1/Tobramycin | 32/64 | 256/256 | 0.185-1 | 2/8 | ↓ x4 | ↓ x3 | Synergism |
| Cx1/Amikacin | 32/1 | 256/4 | 1-4 | nd | nd | nd | Indifference |
| Cx1/Ciprofloxacin | 32/0.5 | 256/4 | 0.5/4 | 8/0.125 | ↓ x2 | ↓ x2 | Additivity |

TABLE XII
Synergism against a mucoid strain of Pseudomonas aeruginosa having a resistance to rifampicin and the
trimethoprim-sulphamethoxazole combination (or PaR5) (n = 4)

| | | | | Optimum | | | |
|---|---|---|---|---|---|---|---|
| | MIC | Ranges tested | | concentrations | Difference | Difference | |
| Combinations | (mg/L) | (mg/L) | FIC index | (mg/L) | Cx1 MIC | ATB MIC | Finding |
| Cx1/Ticarcillin-Clavulanic | 32/32 | 256/512 | 1-4 | nd | nd | nd | Indifference |
| acid | | | | | | | |
| Cx1/Piperacillin-Tazobactam | 32/32 | 256/512 | 0.31-1 | 2/8 | ↓ x4 | ↓ x2 | Synergism |
| Cx1/Ceftazidime | 32/8 | 256/64 | 1-4 | nd | nd | nd | Indifference |
| Cx1/Imipenem | 32/2 | 256/16 | 0.25-1 | 4/0.25 | ↓ x3 | ↓ x3 | Synergism |
| Cx1/Rifampicin | 32/16 | 256/256 | 0.185-1 | 2/2 | ↓ x4 | ↓ x3 | Synergism |
| Cx1/Colimycin | 32/16 | 256/64 | 0.187-1 | 2/2 | ↓ x4 | ↓ x3 | Synergism |
| Cx1/Fosfomycin | 32/16 | 256/256 | 0.375-1 | 8/2 | ↓ x2 | ↓ x3 | Synergism |
| | | | | 4/4 | ↓ x3 | ↓ x2 | |
| Cx1/Tobramycin | 32/1 | 256/4 | 0.25-1 | 4/0.125 | ↓ x3 | ↓ x3 | Synergism |
| Cx1/Amikacin | 32/0.5 | 256/4 | 1-4 | nd | nd | nd | Indifference |
| Cx1/Ciprofloxacin | 32/1 | 256/4 | 0.31-1 | 2/0.25 | ↓ x4 | ↓ x2 | Synergism |

### 5. Identification and Antibiograms

5.1 EcR1: Penicillinase-Producing Escherichia coli without Associated Resistance
The bacterial and antibiogramidentification is carried out by the VITEK 2 system (bioMérieux) and by the disk diffusion technique.

TABLE A1

| | | Dmin | | | |
|---|---|---|---|---|---|
| Antibiotics | Diameter | Dmax | Results | MIC mg/L | Results |
| Amoxicillin | 6 | 14-21 | Resistant | ≧32 | Resistant |
| Amox + | 20 | 14-21 | Intermediate | 4 | Susceptible |
| clavulanic acid | | | | | |
| Ticarcillin | 6 | 18-22 | Resistant | ≧128 | Resistant |
| Piperacillin | 17 | 12-20 | Intermediate | ≦8 | Susceptible |
| Piper + | 25 | 14-21 | Susceptible | ≦4 | Susceptible |
| tazobactam | | | | | |
| C1G | 17 | 12-18 | Intermediate | 4 | Susceptible |
| Cefoxitin | 24 | 15-22 | Susceptible | ≦4 | Susceptible |
| Cefotaxime | 30 | 15-21 | Susceptible | ≦1 | Susceptible |
| Ceftazidime | | | | ≦1 | Susceptible |
| Imipenem | | | | ≦1 | Susceptible |
| Aztreonam | 28 | 17-23 | Susceptible | | |
| Tobramycin | 19 | 14-16 | Susceptible | ≦1 | Susceptible |
| Gentamicin | 20 | 14-16 | Susceptible | ≦1 | Susceptible |
| Amikacin | 19 | 15-17 | Susceptible | ≦2 | Susceptible |
| Netilmicin | 24 | 17-19 | Susceptible | ≦1 | Susceptible |
| Minocycline | 20 | 17-19 | Susceptible | | |
| Colistin | 15 | 15 | Susceptible | | |
| Trimethoprim- | 21 | 10-16 | Susceptible | ≦20 | Susceptible |
| Sulphamet. | | | | | |
| Nalidixic acid | | | | ≦2 | Susceptible |
| Norfloxacin | | | | ≦0.5 | Susceptible |
| Ofloxacin | | | | ≦0.25 | Susceptible |
| Pefloxacin | 26 | 16-22 | Susceptible | | |
| Ciprofloxacin | 27 | 19-22 | Susceptible | ≦0.25 | Susceptible |
| Rifampicin | 16 | 14-19 | Intermediate | | |
| Fosfomycin | 24 | 14 | Susceptible | | |
| Nitrofurantoin | | | | ≦16 | Susceptible |
| Cefepime | 27 | 15-21 | Susceptible | | |

TABLE A2

| | | Cc | | | | | |
|---|---|---|---|---|---|---|---|
| | MIC mg/L | CA-SFM | | Cc | | | |
| | (diameter | 2011 | CA-SFM | EUCAST | EUCAST | Cc CLSI | CLSI |
| Antibiotics | in mm) | mg/L | Interpretation | 2011 | Interpretation | 2011 | Interpretation |
| Amoxicillin | ≧32 | 4-8 | Resistant | 8 | Resistant | 8-32 | Resistant |
| Amox + | 4 | 4-8 | Susceptible | 8 | Susceptible | 8-32 | Susceptible |
| Clavulanic acid | | | | | | | |
| Ticarcillin | ≧128 | 8-16 | Resistant | 8-16 | Resistant | 16-128 | Resistant |
| Piperacillin | ≦8 | 8-16 | Susceptible | 8-16 | Susceptible | 16-128 | Susceptible |
| Piper + | ≦4 | 8-16 | Susceptible | 8-16 | Susceptible | 16-128 | Susceptible |
| tazobactam | | | | | | | |
| Cefalotine | 4 | 8-32 | Susceptible | 16 | Susceptible | 8-32 | Susceptible |
| Cefoxitin | ≦4 | 8-32 | Susceptible | NA | — | 8-32 | Susceptible |
| Cefotaxime | ≦1 | 1-2 | Susceptible | 1-2 | Susceptible | 1-4 | Susceptible |
| Ceftazidime | ≦1 | 1-4 | Susceptible | 1-4 | Susceptible | 4-16 | Susceptible |
| Cefepime | 27 | 24 | Susceptible | 21-24 | Susceptible | 14-18 | Susceptible |
| Imipenem | ≦0.5 | 0.5-1 | Susceptible | 2-8 | Susceptible | 4-16 | Susceptible |
| Aztreonam | 28 | 21-27 | Susceptible | 24-27 | Susceptible | 17-21 | Susceptible |
| Tobramycin | ≦1 | 2-4 | Susceptible | 2-4 | Susceptible | 4-16 | Susceptible |
| Gentamicin | ≦1 | 2-4 | Susceptible | 2-4 | Susceptible | 4-16 | Susceptible |
| Amikacin | ≦2 | 8-16 | Susceptible | 8-16 | Susceptible | 16-64 | Susceptible |
| Netilmicin | ≦1 | 2-4 | Susceptible | 2-4 | Susceptible | 8-32 | Susceptible |
| Minocycline | 20 | 17-19 | Susceptible | — | — | 12-16 | Susceptible |
| Colistin | 15 | 15 | Susceptible | 17 | Resistant | nd | nd |
| Trimethoprim- | ≦20 | 2-4 | Susceptible | 2-4 | Susceptible | 2-4 | Susceptible |
| Sulphamet. | | | | | | | |
| Nalidixic acid | ≦2 | 8-16 | Susceptible | NA | — | 16-32 | Susceptible |
| Norfloxacin | ≦0.5 | 0.5-1 | Susceptible | 0.5-1 | Susceptible | 4-16 | Susceptible |
| Ofloxacin | ≦0.25 | 0.5-1 | Susceptible | 0.5-1 | Susceptible | 2-8 | Susceptible |
| Ciprofloxacin | ≦0.25 | 0.5-1 | Susceptible | — | — | 1-4 | Susceptible |
| Rifampicin | 16 | 14-19 | Intermediate | — | — | nd | nd |
| Fosfomycin | 24 | 14 | Susceptible | — | — | 12-16 | Susceptible |
| Nitrofurantoin | ≦16 | 64 | Susceptible | 64 | Susceptible | 32-128 | Susceptible |

*Cc: Critical concentration

Expert finding July 2006: Penicillinase acquired

The fluctuations relative to the type of associated resistance for certain strains between Tables A1 and A2 are due to the development of the French (CA-SFM), European (EUCAST) and American (CLSI) recommendations for the categorization of bacterial strains.

5.2 EcR2: Cephalosporinase-Hyperproducing Strain of Escherichia coli, Having an Associated Resistance to the Aminoglycosides, Quinolones and the Trimethoprim-Sulphamethoxazole Combination

The bacterial and antibiogramidentification is carried out by the VITEK 1 system (bioMérieux) and by the disk diffusion technique

TABLE B1

| | | Dmin | | |
|---|---|---|---|---|
| Antibiotics | Diameter | Dmax | Results | MIC mg/L |
| Amoxicillin | | | Resistant | ≧32 |
| Amox + clavulanic acid | | | Resistant | ≧32 |
| Ticarcillin | | | Intermediate | 32 |
| C1G | | | Resistant | ≧64 |
| Cefoxitin | 11 | 15-22 | Resistant | |
| Cefotaxime | 24 | 15-21 | Intermediate | |
| Ceftazidime | 20 | 15-21 | Intermediate | |
| Imipenem | | | Susceptible | ≦4 |
| Tobramycin | 10 | 16-18 | Resistant | |
| Gentamicin | 12 | 16-18 | Resistant | |
| Netilmicin | 21 | 19-21 | Susceptible | ≦1 |
| Trimethoprim- | | | Intermediate | 160 |
| Sulphamet. | | | | |
| Nalidixic acid | | | Resistant | ≧32 |
| Pefloxacin | | | Resistant | ≧8 |
| Nitrofurantoin | | | Susceptible | ≦25 |

TABLE B2

| | | Cc* CA- | | | | | |
|---|---|---|---|---|---|---|---|
| | MIC mg/L | SFM | | Cc | | | |
| | (diameter | 2011 | CA-SFM | EUCAST | EUCAST | Cc CLSI | CLSI |
| Antibiotics | in mm) | mg/L | Interpretation | 2011 | Interpretation | 2011 | Interpretation |
| Amoxicillin | ≧32 | 4-8 | Resistant | 8 | Resistant | 8-32 | Resistant |
| Amox + | ≧32 | 4-8 | Resistant | 8 | Resistant | 8-32 | Resistant |
| clavulanic acid | | | | | | | |
| Ticarcillin | 32 | 8-16 | Resistant | 8-16 | Resistant | 16-128 | Resistant |
| Cefalotine | ≧64 | 8-32 | Resistant | 16 | Resistant | 8-32 | Resistant |
| Cefoxitin | 11 | 15-22 | Resistant | 19 | Resistant | 14-18 | Resistant |
| **Cefotaxime | 24 | 23-26 | Intermediate | 18-21 | Resistant | 22-26 | Intermediate |
| **Ceftazidime | 20 | 23-26 | Resistant | 19-22 | Intermediate | 17-21 | Intermediate |
| Imipenem | ≦0.5 | 0.5-1 | Susceptible | 2-8 | Susceptible | 4-16 | Susceptible |
| Tobramycin | 10 | 16-18 | Resistant | 13-16 | Resistant | 12-15 | Resistant |
| **Gentamicin | 12 | 16-18 | Resistant | 14-17 | Resistant | 12-15 | Resistant |
| Netilmicin | ≦1 | 2-4 | Susceptible | 2-4 | Susceptible | 8-32 | Susceptible |
| Trimethoprim - | 160 | 2-4 | Resistant | 2-4 | Resistant | 8-16 | Resistant |
| Sulphamet. | | | | | | | |
| Nalidixic acid | ≧32 | 8-16 | Resistant | — | — | 16-32 | Resistant |
| Pefloxacin | ≧8 | 1-4 | Resistant | — | — | — | — |
| Nitrofurantoin | ≦25 | 64 | Susceptible | 64 | Susceptible | 32-128 | Susceptible |

*Cc: Critical concentration
**Note: the disk load differs between CA-SFM and EUCAST; the EUCAST interpretation is therefore not applicable in the present case. Furthermore, the technique for carrying out the disk diffusion test differs in the two reference standards.

The fluctuations relative to the type of associated resistance for certain strains between Tables B1 and B2 are due to the development of the French (CA-SFM), European (EUCAST) and American (CLSI) recommendations for the categorization of bacterial strains.

5.3 EcR3: ESBL-Producing Escherichia coli Having an Associated Resistance to the Aminoglycosides, Rifampicin and the Trimethoprim-Sulphamethoxazole Combination

The bacterial and antibiogramidentification is carried out by the VITEK 2 system (bioMerieux) and by the disk diffusion technique

TABLE C1

| | | Dmin | |
|---|---|---|---|
| Antibiotics | Diameter | Dmax | Results |
| Amoxicillin | 6 | 14-21 | Resistant |
| Amox + clavulanic acid | 16 | 14-21 | Intermediate |
| Ticarcillin | 6 | 18-22 | Resistant |
| Piperacillin | 13 | 12-20 | Intermediate |
| Piper + tazobactam | | | Intermediate |
| C1G | 6 | 12-18 | Resistant |
| Cefoxitin | | | Intermediate |
| Cefotaxime | 19 | 15-21 | Intermediate |
| Imipenem | 27 | 17-22 | Susceptible |
| Tobramycin | 6 | 14-16 | Resistant |
| Gentamicin | 12 | 14-16 | Resistant |
| Amikacin | 17 | 15-17 | Susceptible |
| Netilmicin | 10 | 17-19 | Resistant |
| Minocycline | 21 | 17-19 | Susceptible |
| Colistin | 16 | 15 | Susceptible |
| Trimethoprim- | 6 | 10-16 | Resistant |
| Sulphamet. | | | |
| Pefloxacin | 23 | 16-22 | Susceptible |
| Ciprofloxacin | 25 | 19-22 | Susceptible |
| Rifampicin | 15 | 14-19 | Intermediate |
| Fosfomycin | 23 | 14 | Susceptible |
| Cefepime | 24 | 15-21 | Intermediate |

TABLE C2

| | | Cd* | | Cd | | Cd | |
|---|---|---|---|---|---|---|---|
| | diameter | CA-SFM | CA-SFM | EUCAST | EUCAST | CLSI | CLSI |
| Antibiotics | in mm | 2011 | Interpretation | 2011 | Interpretation | 2011 | Interpretation |
| Amoxicillin | 6 | 16-19 | Resistant | 14 | Resistant | 13-17 | Resistant |
| Amox + | 16 | 16-21 | Intermediate | 17 | Resistant | 13-18 | Intermediate |
| clavulanic acid | | | | | | | |
| Ticarcillin | 6 | 22-24 | Resistant | 22-23 | Resistant | 14-20 | Resistant |
| Piperacillin | 13 | 16-20 | Resistant | 15-18 | Resistant | 17-21 | Resistant |
| **Piper + | 18 | 17-21 | Intermediate | 15-18 | Susceptible | 17-21 | Intermediate |
| tazobactam | | | | | | | |
| Cefalotine | 6 | 12-18 | Resistant | — | — | 14-18 | Resistant |
| Cefoxitin | 6 | 15-22 | Resistant | 19 | Resistant | 14-18 | Resistant |
| **Cefotaxime | 19 | 23-26 | Resistant | 18-21 | Intermediate | 22-26 | Resistant |
| Cefepime | 24 | 24 | Susceptible | 21-24 | Susceptible | 14-18 | Susceptible |
| Imipenem | 27 | 17-24 | Susceptible | 15-21 | Susceptible | 13-16 | Susceptible |
| Tobramycin | 6 | 16-18 | Resistant | 13-16 | Resistant | 12-15 | Resistant |
| **Gentamicin | 12 | 16-18 | Resistant | 14-17 | Resistant | 12-15 | Intermediate |
| Amikacin | 17 | 15-17 | Susceptible | 13-16 | Susceptible | 14-17 | Susceptible |
| **Netilmicin | 10 | 19-21 | Resistant | 12-15 | Resistant | 12-15 | Resistant |
| Minocycline | 21 | 17-19 | Susceptible | — | — | 12-16 | Susceptible |
| Colistin | 16 | 15 | Susceptible | — | — | — | — |
| Trimethoprim - | 6 | 13-16 | Resistant | 13-16 | Resistant | 10-16 | Resistant |
| Sulphamet. | | | | | | | |
| Pefloxacin | 23 | 16-22 | Susceptible | — | — | — | — |
| Ciprofloxacin | 25 | 22-25 | Susceptible | — | — | 15-21 | Susceptible |
| Rifampicin | 15 | 14-19 | Intermediate | — | — | — | — |
| Fosfomycin | 23 | 14 | Susceptible | — | — | 12-16 | Susceptible |

*Cd: Critical diameter
**Note: the disk load differs between CA-SFM and EUCAST; the EUCAST interpretation is therefore not applicable in the present case. Furthermore, the technique for carrying out the disk diffusion test differs in the two reference standards.

The fluctuations relative to the type of associated resistance for certain strains between Tables C1 and C2 are due to the development of the French (CA-SFM), European (EUCAST) and American (CLSI) recommendations for the categorization of bacterial strains.

5.4 SaR1: Meticillin-Resistant Staphylococcus aureus without Associated Resistance

The bacterial and antibiogramidentification is carried out by the VITEK 2 system (bioMérieux)

TABLE D1

| | | Dmin | | | |
|---|---|---|---|---|---|
| Antibiotics | Diameter | Dmax | Results | MIC mg/L | Results |
| Penicillin G | 28 | 9-29 | Resistant | ≧0.5 | Resistant |
| Oxacillin | | | | ≧8 | Resistant |
| Kanamycin | 20 | 15-17 | Susceptible | ≦4 | Susceptible |
| Tobramycin | 22 | 14-16 | Susceptible | ≦1 | Susceptible |
| Gentamicin | 23 | 14-16 | Susceptible | ≦0.5 | Susceptible |
| Chloramphenicol | 25 | 19-23 | Susceptible | | |
| Minocycline | 27 | 17-19 | Susceptible | | |
| Erythromycin | 25 | 17-22 | Susceptible | ≦0.25 | Susceptible |
| Lincomycin | 24 | 17-21 | Susceptible | ≦1 | Susceptible |
| Pristinamycin | 25 | 19-22 | Susceptible | ≦0.5 | Susceptible |
| Quinupristin- | | | | ≦0.25 | Susceptible |
| dalfopristin | | | | | |
| Trimethoprim- | 26 | 10-16 | Susceptible | ≦10 | Susceptible |
| Sulphamet. | | | | | |
| Ofloxacin | 24 | 16-22 | Susceptible | 1 | Susceptible |
| Fusidic acid | 28 | 15-22 | Susceptible | ≦0.5 | Susceptible |
| Vancomycin | | | Susceptible | ≦1 | Susceptible |
| Teicoplanin | | | Susceptible | ≦0.5 | Susceptible |
| Rifampicin | 30 | 14-29 | Susceptible | ≦0.5 | Susceptible |
| Fosfomycin | 40 | 14 | Susceptible | ≦8 | Susceptible |
| Linezolid | 29 | 24-28 | Susceptible | 2 | Susceptible |
| Minocycline | | | | ≦0.5 | Susceptible |
| Nitrofurantoin | | | | ≦16 | Susceptible |

TABLE D2

| | | Cc* CA- | | | | | |
|---|---|---|---|---|---|---|---|
| | | SFM | | Cc | | Cc | |
| | MIC | 2011 | CA-SFM | EUCAST | EUCAST | CLSI | CLSI |
| Antibiotics | mg/L | mg/L | Interpretation | 2011 | Interpretation | 2011 | Interpretation |
| Penicillin G | ≧0.5 | 0.12 | Resistant | 0.125 | Resistant | 0.12-0.25 | Resistant |
| Oxacillin | ≧8 | 2 | Resistant | 2 | Resistant | 2-4 | Resistant |
| Kanamycin | ≦4 | 8-16 | Susceptible | 8-16 | Susceptible | 16-64 | Susceptible |
| Tobramycin | ≦1 | 1 | Susceptible | 1 | Susceptible | 4-16 | Susceptible |
| Gentamicin | ≦0.5 | 1 | Susceptible | 1 | Susceptible | 4-16 | Susceptible |
| Erythromycin | ≦0.25 | 1-2 | Susceptible | 1-2 | Susceptible | 0.5-8 | Susceptible |
| Lincomycin | ≦1 | 2-8 | Susceptible | — | — | — | — |
| Pristinamycin | ≦0.5 | 1-2 | Susceptible | — | — | — | — |
| Quinupristin-dalfopristin | ≦0.25 | 1-2 | Susceptible | 1-2 | Susceptible | 1-4 | Susceptible |
| Trimethoprim - | ≦10 | 2-4 | Susceptible | 2-4 | Susceptible | 2-4 | Susceptible |
| Sulphamet. | | | | | | | |
| Ofloxacin | 1 | 1 | Susceptible | 1 | Susceptible | 1-4 | Susceptible |
| Fusidic acid | ≦0.5 | 1 | Susceptible | 1 | Susceptible | — | — |
| Vancomycin | ≦1 | 2 | Susceptible | 2 | Susceptible | 4-32 | Susceptible |
| Teicoplanin | ≦0.5 | 4 | Susceptible | 2 | Susceptible | 8-32 | Susceptible |
| Rifampicin | ≦0.5 | 0.06-0.5 | Susceptible | 0.064-0.5 | Susceptible | 1-4 | Susceptible |
| Fosfomycin | ≦8 | 32 | Susceptible | 32 | Susceptible | — | — |
| Linezolid | 2 | 4 | Susceptible | 4 | Susceptible | 4-8 | Susceptible |
| Minocycline | ≦0.5 | 0.5-1 | Susceptible | 0.5-1 | Susceptible | 4-16 | Susceptible |
| Nitrofurantoin | ≦16 | 64 | Susceptible | 64 | Susceptible | 32-128 | Susceptible |

*Cc: Critical concentration

February 2006: Detection of the gene mecA by the PCR technique: POSITIVE
Expert finding July 2006: totally typical phenotype: modification of the PLPs
The fluctuations relative to the type of associated resistance for certain strains between Tables D1 and D2 are due to the development of the French (CA-SFM), European (EUCAST) and American (CLSI) recommendations for the categorization of bacterial strains.

5.5 SaR3: Meticillin-Resistant Staphylococcus aureus Having an Associated Resistance to the Aminoglycosides and Fluoroquinolones

The bacterial and antibiogramidentification is carried out by the VITEK 2 system (bioMérieux) and by the disk diffusion technique

TABLE E1

| Antibiotics | Diameter | Dmin Dmax | Results |
|---|---|---|---|
| Penicillin G | 26 | 9-29 | Resistant |
| Oxacillin | | | |
| Kanamycin | 6 | 15-17 | Resistant |
| Tobramycin | 6 | 14-16 | Resistant |
| Gentamicin | 20 | 14-16 | Susceptible |
| Chloramphenicol | 6 | 19-23 | Resistant |
| Minocycline | 26 | 17-19 | Susceptible |
| Erythromycin | 25 | 17-22 | Susceptible |
| Lincomycin | 23 | 17-21 | Susceptible |
| Pristinamycin | 23 | 19-22 | Susceptible |
| Trimethoprim-Sulphamet. | 27 | 10-16 | Susceptible |
| Ofloxacin | 6 | 16-22 | Resistant |
| Fusidic acid | 29 | 15-22 | Susceptible |
| Vancomycin | | | Susceptible |
| Teicoplanin | | | Susceptible |
| Rifampicin | 31 | 14-29 | Susceptible |
| Fosfomycin | 26 | 14 | Susceptible |
| Linezolid | 26 | 24-28 | Susceptible |

TABLE E2

| | | Cd* | | Cd | | Cd | |
|---|---|---|---|---|---|---|---|
| | diameter | CA-SFM | CA-SFM | EUCAST | EUCAST | CLSI | CLSI |
| Antibiotics | in mm | 2011 | Interpretation | 2011 | Interpretation | 2011 | Interpretation |
| Penicillin G | 26 | | Resistant | 26 | Resistant | 28-29 | Resistant |
| Cefoxitin | 17 | 25-37 | Resistant | 22 | Resistant | 21-22 | Resistant |
| **Kanamycin | 6 | 15-17 | Resistant | 16-18 | Resistant | 13-18 | Resistant |
| Tobramycin | 6 | 20 | Resistant | 18 | Resistant | 12-15 | Resistant |
| **Gentamicin | 20 | 20 | Susceptible | 18 | Susceptible | 12-15 | Susceptible |
| Erythromycin | 25 | 19-22 | Susceptible | 18-21 | Susceptible | 13-23 | Susceptible |
| Lincomycin | 23 | 17-21 | Susceptible | — | — | — | — |
| Pristinamycin | 23 | 19-22 | Susceptible | — | — | — | — |
| Trimethoprim - | 27 | 13-16 | Susceptible | 14-17 | Susceptible | 10-16 | Susceptible |
| Sulphamet. | | | | | | | |
| Ofloxacin | 6 | 22 | Resistant | 20 | Resistant | 14-18 | Resistant |
| Fusidic acid | 29 | 24 | Susceptible | 24 | Susceptible | — | — |
| Vancomycin | 28 | 17 | Susceptible | — | — | — | — |
| Teicoplanin | 27 | 17 | Susceptible | — | — | 10-14 | Susceptible |
| Rifampicin | 31 | 24-29 | Susceptible | 23-26 | Susceptible | 16-20 | Susceptible |
| Fosfomycin | 26 | 14 | Susceptible | — | — | — | — |
| **Linezolid | 26 | 24 | Susceptible | 19 | Susceptible | 20-21 | Susceptible |
| Minocycline | 26 | 21-23 | Susceptible | 20-23 | Susceptible | 14-19 | Susceptible |

*Cd: Critical diameter
**Note: the disk load differs between CA-SFM and EUCAST; the EUCAST interpretation is therefore not applicable in the present case. Furthermore, the technique for carrying out the disk diffusion test differs in the two reference standards.

The fluctuations relative to the type of associated resistance for certain strains between Tables E1 and E2 are due to the development of the French (CA-SFM), European (EUCAST) and American (CLSI) recommendations for the categorization of bacterial strains.

5.6 SaR4: Meticillin-Resistant Staphylococcus aureus Having an Associated Resistance to the Aminoglycosides, Fluoroquinolones, Macrolides-Lincosamines-Synergistins and Ofloxacin

The bacterial and antibiogramidentification is carried out by the VITEK 2 system (bioMérieux) and by the disk diffusion technique

TABLE F1

| Antibiotics | Diameter | Dmin Dmax | Results | MIC mg/L |
|---|---|---|---|---|
| Penicillin G | | | Resistant | |
| Oxacillin | | | Resistant | ≧8 |
| Kanamycin | 7 | 15-17 | Resistant | |
| Tobramycin | 6 | 20-20 | Resistant | |
| Gentamicin | 21 | 20-20 | Susceptible | |
| Chloramphenicol | | | Susceptible | 8 |
| Tetracycline | | | Susceptible | ≦1 |
| Minocycline | | | Susceptible | ≦4 |
| Erythromycin | | | Resistant | ≧8 |
| Lincomycin | | | Resistant | ≧16 |
| Pristinamycin | | | Susceptible | ≦2 |
| Trimethoprim- | | | Susceptible | ≦10 |
| Sulphamet. | | | | |
| Ofloxacin | | | Resistant | ≧8 |
| Nitrofurantoin | | | Susceptible | ≦25 |
| Fusidic acid | | | Susceptible | ≦1 |
| Vancomycin | | | Susceptible | 1 |
| Teicoplanin | | | Susceptible | ≦4 |
| Rifampicin | | | Susceptible | ≦1 |
| Fosfomycin | | | Resistant | ≧64 |

TABLE F2

| | MIC mg/L | Cc* CA-SFM | | Cc | | | |
|---|---|---|---|---|---|---|---|
| | (diameter | 2011 | CA-SFM | EUCAST | EUCAST | Cc CLSI | CLSI |
| Antibiotics | in mm) | mg/L | Interpretation | 2011 | Interpretation | 2011 | Interpretation |
| Penicillin G | | | Resistant | | Resistant | 0.12-0.25 | Resistant |
| Oxacillin | ≧8 | 2 | Resistant | 2 | Resistant | 2-4 | Resistant |
| **Kanamycin | 7 | 15-17 | Resistant | 16-18 | Resistant | 13-18 | Resistant |
| Tobramycin | 6 | 20 | Resistant | 18 | Resistant | 12-15 | Resistant |
| **Gentamicin | 21 | 20 | Susceptible | 18 | Susceptible | 12-15 | Susceptible |
| Tetracycline | ≦1 | 1-2 | Susceptible | 1-2 | Susceptible | 4-16 | Susceptible |
| Minocycline | ≦0.5 | 0.5-1 | Susceptible | 0.5-1 | Susceptible | 4-16 | Susceptible |
| Erythromycin | ≧8 | 1-2 | Resistant | 1-2 | Resistant | 0.5-8 | Resistant |
| Lincomycin | ≧16 | 2-8 | Resistant | — | — | — | — |
| Pristinamycin | ≦2 | 1-2 | Susceptible | — | — | — | — |
| Trimethoprim - | ≦10 | 2-4 | Susceptible | 2-4 | Susceptible | 2-4 | Susceptible |
| Sulphamet. | | | | | | | |
| Ofloxacin | ≧8 | 1 | Resistant | 1 | Resistant | 1-4 | Resistant |
| Nitrofurantoin | ≦25 | 64 | Susceptible | 64 | Susceptible | 32-128 | Susceptible |
| Fusidic acid | ≦1 | 1 | Susceptible | 1 | Susceptible | — | — |
| Vancomycin | 1 | 2 | Susceptible | 2 | Susceptible | 4-32 | Susceptible |
| Teicoplanin | ≦4 | 4 | Susceptible | 2 | Susceptible | 8-32 | Susceptible |
| Rifampicin | ≦1 | 0.06-0.5 | Susceptible | 0.064-0.5 | Susceptible | 1-4 | Susceptible |
| Fosfomycin | ≧64 | 32 | Resistant | 32 | Resistant | — | — |

*Cc: Critical concentration
**Note: the disk load differs between CA-SFM and EUCAST; the EUCAST interpretation is therefore not applicable in the present case. Furthermore, the technique for carrying out the disk diffusion test differs in the two reference standards.

The fluctuations relative to the type of associated resistance for certain strains between Tables F1 and F2 are due to the development of the French (CA-SFM), European (EUCAST) and American (CLSI) recommendations for the categorization of bacterial strains.

5.7 PaR2: Pseudomonas aeruginosa Having an Associated Resistance to the β-Lactams, the Trimethoprim-Sulphamethoxazole Combination and Fosfomycin

The bacterial and antibiogramidentification is carried out by the VITEK 2 system (bioMérieux) and by the disk diffusion technique

TABLE G1

| Antibiotics | Diameter | Dmin-Dmax | Results |
|---|---|---|---|
| Ticar + clavulanic acid | 10 | 18-22 | Resistant |
| Ticarcillin | 10 | 18-22 | Resistant |
| Piperacillin | 22 | 12-18 | Susceptible |
| Piper + tazobactam | 22 | 14-19 | Susceptible |
| Ceftazidime | 23 | 15-21 | Susceptible |
| Aztreonam | 16 | 17-23 | Resistant |
| Imipenem | 27 | 17-22 | Susceptible |
| Tobramycin | 24 | 14-16 | Susceptible |
| Gentamicin | 21 | 14-16 | Susceptible |
| Amikacin | 22 | 15-17 | Susceptible |
| Netilmicin | 19 | 17-19 | Susceptible |
| Minocycline | 6 | 17-19 | Resistant |
| Colistin | 20 | 15-15 | Susceptible |
| Trimethoprim- | 6 | 10-16 | Resistant |
| Sulphamet. | | | |
| Pefloxacin | 8 | 16-22 | Resistant |
| Rifampicin | 25 | 19-22 | Susceptible |
| Fosfomycin | 13 | 14-19 | Resistant |
| Ciprofloxacin | 18 | 14-14 | Susceptible |
| Cefepime | 19 | 15-21 | Intermediate |

TABLE G2

| | | Cd* | | Cd | | Cd | |
|---|---|---|---|---|---|---|---|
| | diameter | CA-SFM | CA-SFM | EUCAST | EUCAST | CLSI | CLSI |
| Antibiotics | in mm | 2011 | Interpretation | 2011 | Interpretation | 2011 | Interpretation |
| Ticar + | 10 | 22 | Resistant | 17 | Resistant | 14-15 | Resistant |
| clavulanic acid | | | | | | | |
| Ticarcillin | 10 | 22 | Resistant | 17 | Resistant | 14-15 | Resistant |
| **Piperacillin | 22 | 18 | Susceptible | 19 | Susceptible | 17-18 | Susceptible |
| **Piper + | 22 | 19 | Susceptible | 19 | Susceptible | 17-18 | Susceptible |
| tazobactam | | | | | | | |
| **Ceftazidime | 23 | 19 | Susceptible | 16 | Susceptible | 14-18 | Susceptible |
| Cefepime | 19 | 19 | Susceptible | 18 | Susceptible | 14-18 | Susceptible |
| Aztreonam | 16 | 19-27 | Resistant | 16-50 | Resistant | 15-22 | Intermediate |
| Imipenem | 27 | 17-22 | Susceptible | 17-20 | Susceptible | 13-16 | Susceptible |
| Tobramycin | 24 | 16 | Susceptible | 16 | Susceptible | 12-15 | Susceptible |
| **Gentamicin | 21 | 16 | Susceptible | 15 | Susceptible | 12-15 | Susceptible |
| Amikacin | 22 | 15-17 | Susceptible | 15-18 | Susceptible | 14-17 | Susceptible |
| **Netilmicin | 19 | 19 | Susceptible | 12 | Susceptible | 12-15 | Susceptible |
| Minocycline | 6 | — | Resistant | — | — | — | — |
| Colistin | 20 | — | Susceptible | — | — | 10-11 | Susceptible |
| Trimethoprim- | 6 | — | Resistant | 16 | Resistant | — | — |
| Sulphamet. | | | | | | | |
| Ciprofloxacin | 25 | 22-25 | Susceptible | 22-25 | Susceptible | 15-21 | Susceptible |
| Rifampicin | 25 | 14-19 | Susceptible | — | — | — | — |
| Fosfomycin | 13 | 14 | Resistant | — | — | — | — |

*Cd: Critical diameter
**Note: the disk load differs between CA-SFM and EUCAST; the EUCAST interpretation is therefore not applicable in the present case. Furthermore, the technique for carrying out the disk diffusion test differs in the two reference standards.

The fluctuations relative to the type of associated resistance for certain strains between Tables G1 and G2 are due to the development of the French (CA-SFM), European (EUCAST) and American (CLSI) recommendations for the categorization of bacterial strains.

5.8 PaR3: Pseudomonas aeruginosa Having an Associated Resistance to the β-Lactams, the Aminoglycosides (Including the Carbapenems), the Trimethoprim-Sulphamethoxazole Combination and Ciprofloxacin

The bacterial and antibiogramidentification is carried out by the VITEK 2 system (bioMérieux)

TABLE H1

| Antibiotics | MIC | Results |
|---|---|---|
| Ticar + clavulanic acid | ≧128 | Resistant |
| Ticarcillin | ≧128 | Resistant |
| Piperacillin | ≧128 | Resistant |
| Piper + tazobactam | ≧128 | Resistant |
| Ceftazidime | 4 | Susceptible |
| Aztreonam | 16 | Susceptible |
| Imipenem | ≧16 | Resistant |
| Meropenem | ≧16 | Resistant |
| Tobramycin | ≧16 | Resistant |
| Gentamicin | ≧16 | Resistant |
| Amikacin | 4 | Susceptible |
| Minocycline | 4 | Resistant |
| Colistin | ≦0.5 | Susceptible |
| Trimethoprim- | | |
| Sulphamet. | ≧320 | Resistant |
| Pefloxacin | 4 | Intermediate |
| Ciprofloxacin | 1 | Susceptible |
| Cefepime | 16 | Intermediate |

TABLE H2

| | | Cc* CA-SFM | | Cc | | | |
|---|---|---|---|---|---|---|---|
| | MIC | 2011 | CA-SFM | EUCAST | EUCAST | Cc CLSI | CLSI |
| Antibiotics | mg/L | mg/L | Interpretation | 2011 | Interpretation | 2011 | Interpretation |
| Ticar + | ≧128 | 16 | Resistant | 16 | Resistant | 64-128 | Resistant |
| clavulanic acid | | | | | | | |
| Ticarcillin | ≧128 | 16 | Resistant | 16 | Resistant | 64-128 | Resistant |
| Piperacillin | ≧128 | 16 | Resistant | 16 | Resistant | 64-128 | Resistant |
| Piper + | ≧128 | 16 | Resistant | 16 | Resistant | 64-128 | Resistant |
| tazobactam | | | | | | | |
| Ceftazidime | 4 | 8 | Susceptible | 8 | Susceptible | 8-32 | Susceptible |
| Cefepime | 16 | 8 | Resistant | 8 | Resistant | 8-32 | Intermediate |
| Aztreonam | 16 | 1-16 | Resistant | 1-16 | Resistant | 8-32 | Intermediate |
| Imipenem | ≧16 | 4-8 | Resistant | 4-8 | Resistant | 4-16 | Resistant |
| Meropenem | ≧16 | 2-8 | Resistant | 2-8 | Resistant | 4-16 | Resistant |
| Tobramycin | ≧16 | 4 | Resistant | 4 | Resistant | 4-16 | Resistant |
| Gentamicin | ≧16 | 4 | Resistant | 4 | Resistant | 4-16 | Resistant |
| Amikacin | 4 | 8-16 | Susceptible | 8-16 | Susceptible | 16-64 | Susceptible |
| Minocycline | 4 | — | Resistant | — | — | — | — |
| Colistin | ≦0.5 | 2-4 | Susceptible | 4 | Susceptible | 2-8 | Susceptible |
| Trimethoprim - | ≧320 | — | Resistant | 4 | Resistant | — | — |
| Sulphamet. | | | | | | | |
| Ciprofloxacin | 1 | 0.5-1 | Intermediate | 0.5-1 | Intermediate | 1-4 | Susceptible |

*Cc: critical concentration

The fluctuations relative to the type of associated resistance for certain strains between Tables H1 and H2 are due to the development of the French (CA-SFM), European (EUCAST) and American (CLSI) recommendations for the categorization of bacterial strains.

5.9 PaR5: Pseudomonas aeruginosa Having an Associated Resistance to Rifampicin and the Trimethoprim-Sulphamethoxazole Combination

The bacterial and antibiogramidentification is carried out by the VITEK 2 system (bioMérieux) and by the disk diffusion technique

TABLE I1

| Antibiotics | Diameter | Dmin-Dmax | Results |
|---|---|---|---|
| Ticar + clavulanic acid | 40 | 18-22 | Susceptible |
| Ticarcillin | 32 | 18-22 | Susceptible |
| Piperacillin | 33 | 12-18 | Susceptible |
| Piper + tazobactam | 40 | 14-19 | Susceptible |
| Ceftazidime | 32 | 15-21 | Susceptible |
| Aztreonam | 33 | 17-23 | Susceptible |
| Imipenem | 25 | 17-22 | Susceptible |
| Meropenem | 34 | 15-20 | Susceptible |
| Tobramycin | 28 | 14-16 | Susceptible |
| Gentamicin | 25 | 14-16 | Susceptible |
| Amikacin | 27 | 15-17 | Susceptible |
| Netilmicin | 28 | 17-19 | Susceptible |
| Minocycline | 11 | 17-19 | Resistant |
| Colistin | 23 | 15 | Susceptible |
| Trimethoprim- | 6 | 10-16 | Resistant |
| Sulphamet. | | | |
| Norofloxacin | 17 | 22-25 | Resistant |
| Ofloxacin | 11 | 22-25 | Resistant |
| Ciprofloxacin | 30 | 19-22 | Susceptible |
| Rifampicin | 13 | 14-19 | Resistant |
| Fosfomycin | 30 | 14 | Susceptible |
| Cefepime | 32 | 15-21 | Susceptible |

TABLE I2

| | | Cd* | | Cd | | | |
|---|---|---|---|---|---|---|---|
| | diameter | CA-SFM | CA-SFM | EUCAST | EUCAST | Cd CLSI | CLSI |
| Antibiotics | in mm | 2011 | Interpretation | 2011 | Interpretation | 2011 | Interpretation |
| Ticar + | 40 | 22 | Susceptible | 17 | Susceptible | 14-15 | Susceptible |
| clavulanic acid | | | | | | | |
| Ticarcillin | 32 | 22 | Susceptible | 17 | Susceptible | 14-15 | Susceptible |
| **Piperacillin | 33 | 18 | Susceptible | 19 | Susceptible | 17-18 | Susceptible |
| **Piper + | 40 | 19 | Susceptible | 19 | Susceptible | 17-18 | Susceptible |
| tazobactam | | | | | | | |
| **Ceftazidime | 32 | 19 | Susceptible | 16 | Susceptible | 14-18 | Susceptible |
| Cefepime | 32 | 19 | Susceptible | 18 | Susceptible | 14-18 | Susceptible |
| Aztreonam | 33 | 19-27 | Susceptible | 16-50 | Intermediate | 15-22 | Susceptible |
| Imipenem | 25 | 17-22 | Susceptible | 17-20 | Susceptible | 13-16 | Susceptible |
| Meropenem | 34 | 15-22 | Susceptible | 18-24 | Susceptible | 13-16 | Susceptible |
| Tobramycin | 28 | 16 | Susceptible | 16 | Susceptible | 12-15 | Susceptible |
| **Gentamicin | 25 | 16 | Susceptible | 15 | Susceptible | 12-15 | Susceptible |
| Amikacin | 27 | 15-17 | Susceptible | 15-18 | Susceptible | 14-17 | Susceptible |
| **Netilmicin | 28 | 19 | Susceptible | 12 | Susceptible | 12-15 | Susceptible |
| Minocycline | 11 | — | Resistant | — | — | — | — |
| Colistin | 23 | — | Susceptible | — | — | 10-11 | Susceptible |
| Trimethoprim - | 6 | — | Resistant | 16 | Resistant | — | — |
| Sulphamet. | | | | | | | |
| Ciprofloxacin | 30 | 22-25 | Susceptible | 22-25 | Susceptible | 15-21 | Susceptible |
| Rifampicin | 13 | 14-19 | Resistant | — | — | — | — |
| Fosfomycin | 30 | 14 | Susceptible | — | — | — | — |

*Cd: Critical diameter
**Note: the disk load differs between CA-SFM and EUCAST; the EUCAST interpretation is therefore not applicable in the present case. Furthermore, the technique for carrying out the disk diffusion test differs in the two reference standards.

The fluctuations relative to the type of associated resistance for certain strains between Tables I1 and I2 are due to the development of the French (CA-SFM), European (EUCAST) and American (CLSI) recommendations for the categorization of bacterial strains.
6. Synergism of the Combination of Cx1 with Antiseptics Against the Wild-Type Bacterial Strains

6.1. Synergism Against the E. Coli Strain ATCC 25922 (Wild Type)

| | Initial | Ranges | | Optimum | | | |
|---|---|---|---|---|---|---|---|
| | MICs | tested | FIC | MICs | Difference | Difference | |
| Combinations | (mg/L) | (mg/L) | index | (mg/L) | Cx1 MIC | ATS MIC | Finding |
| Cx1/Hexamidine | 4/8 | 64/32 | >1 | nd | nd | nd | Indifference |
| Cx1/Chlorhexidine | 4/<1 | 64/4 | >1 | nd | nd | nd | Indifference |

6.2 Synergism Against the S. Aureus Strain ATCC 29213 (Wild Type)

| | Initial | Ranges | | Optimum | | | |
|---|---|---|---|---|---|---|---|
| | MICs | tested | FIC | MICs | Difference | Difference | |
| Combinations | (mg/L) | (mg/L) | index | (mg/L) | Cx1 MIC | ATS MIC | Finding |
| Cx1/Hexamidine | 8/<1 | 64/4 | >1 | nd | nd | nd | Indifference |
| Cx1/Chlorhexidine | 8/<1 | 64/4 | >1 | nd | nd | nd | Indifference |

6.3. Synergism Against the P. Aeruginosa Strain ATCC 27853 (Wild Type)

| | Initial | Ranges | | Optimum | | | |
|---|---|---|---|---|---|---|---|
| | MICs | tested | FIC | MICs | Difference | Difference | |
| Combinations | (mg/L) | (mg/L) | index | (mg/L) | Cx1 MIC | ATS MIC | Finding |
| Cx1/Hexamidine | 32/32 | 64/256 | 3 | nd | nd | nd | Indifference |
| Cx1/Chlorhexidine | 32/4 | 64/32 | 1.5 | nd | nd | nd | Indifference |

1. Product comprising at least one given antibiotic and a calixarene represented by Formula I below: 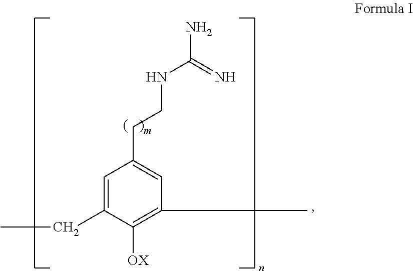
in which:
(i) n=an integer from 4 to 16,
(ii) m=an integer from 1 to 10,
(iii) X is chosen from:
a hydrogen,
an alkyl group, the number of carbons being from 1 to 20, in particular from 1 to 10,
a halogen chosen from Cl, Br, I, or
an amphiphilic group chosen from an anionic group, such as the carboxylates —RCO₂—, the sulphates —RSO₄—, the sulphonates —RSO₃—, a cationic group, such as RNH₃⁺, in which R is an alkyl group, the number of carbons being from 1 to 20, in particular from 1 to 10,
for its use as medicament.

2. The product according to claim 1, in which the calixarene corresponds to the calixarene represented by Formula II below:

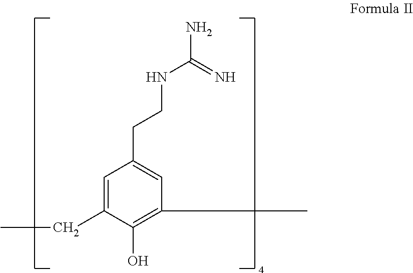

3. The product according to claim 1, in which said given antibiotic is chosen from the group constituted by the β-lactams, the aminoglycosides, fluoroquinolones, fosfomycin, colimycin, rifampicin, tigecycline, or fusidic acid.

4. The product according to claim 3, in which said given antibiotic is chosen from imipenem, piperacillin-tazobactam, penicillin G, cefotaxime, ceftazidime, tobramycin, gentamicin, ciprofloxacin, rifampicin, fosfomycin, colimycin, streptomycin, ticarcillin-clavulanic acid, tigecycline or fusidic acid.

5. A method of treating pathologies involving a bacterial strain having a resistance to at least one defined antibiotic, comprising administering to a subject in need thereof an effective amount of the product according to claim 1.

6. The method according to claim 5, wherein the pathologies involves a resistant bacterial strain belonging to a species chosen from Escherichia coli, Pseudomonas aeruginosa, and Staphylococcus aureus.

7. The method according to claim 6, wherein the pathologies involves a resistant bacterial strain chosen from:
a wild-type strain of Staphylococcus aureus,
a Methicillin-Resistant Staphylococcus aureus strain (MRSA) without associated resistance,
an MRSA strain having a resistance to the aminoglycosides and fluoroquinolones,
an MRSA strain having a resistance to the aminoglycosides, fluoroquinolones, macrolides-lincosamides-synergistins and ofloxacin,
a wild-type strain of Escherichia coli,
an ESBL (Extended-Spectrum β-Lactamase)-producing strain of Escherichia coli having an associated resistance to the aminoglycosides, rifampicin and the trimethoprime-sulphamethoxazole combination,
a penicillinase-producing strain of Escherichia coli without associated resistance,
a cephalosporinase-hyperproducing strain of Escherichia coli, having an associated resistance to the aminoglycosides, quinolones and the trimethoprime-sulphamethoxazole combination,
a wild-type strain of Pseudomonas aeruginosa,
a Pseudomonas aeruginosa strain having a resistance to the β-lactams, the trimethoprime-sulphamethoxazole combination and fosfomycin,
a Pseudomonas aeruginosa strain having a resistance to the β-lactams (including the carbapenems), the aminoglycosides, the trimethoprime-sulphamethoxazole combination and ciprofloxacin,
a mucoid strain of Pseudomonas aeruginosa having a resistance to rifampicin and the trimethoprime-sulphamethoxazole combination.

8. The method according to claim 5, wherein the pathologies are selected from the group consisting of nosocomial and/or community-acquired infections, such as abdominal infections, digestive infections, urinary infections, respiratory infections, neuro-meningeal infections, oro-pharyngeal infections, genital infections, endocarditis, infections of the skin and of the soft tissues, osteo-articular infections, ocular infections, septicaemia and bacteraemia.

9. Pharmaceutical composition comprising as active substance at least one product according to claim 1 in combination with a pharmaceutically acceptable vehicle.

10. Product containing:
a given antibiotic chosen from the group constituted by the β-lactams, the aminoglycosides, fluoroquinolones, fosfomycin, colimycin, rifampicin, tigecycline, or fusidic acid, and
a calixarene represented by Formula I below: 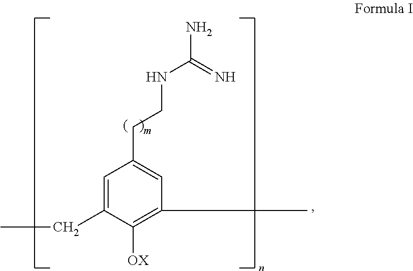
in which:
(i) n=an integer from 4 to 16,
(ii) m=an integer from 1 to 10,
(iii) X is chosen from:
a hydrogen,
an alkyl group, the number of carbons being from 1 to 20, in particular from 1 to 10,
a halogen chosen from Cl, Br, I, or
an amphiphilic group chosen from an anionic group, such as the carboxylates —RCO₂—, the sulphates —RSO₄—, the sulphonates —RSO₃—, a cationic group, such as RNH₃⁺, in which R is an alkyl group, the number of carbons being from 1 to 20, in particular from 1 to 10,
as a combination product, for simultaneous or separate use or spread over time for the treatment of pathologies involving at least one bacterial strain having a resistance to at least one defined antibiotic, such as abdominal infections, digestive infections, urinary infections, respiratory infections, neuro-meningeal infections, infections of the oro-pharyngeal sphere, genital infections, endocarditis, infections of the skin and soft tissues, osteoarticular infections, ocular infections, septicaemia or bacteriaemia.

11. Combination product for use according to claim 10, in which the calixarene corresponds to the calixarene represented by Formula II below:

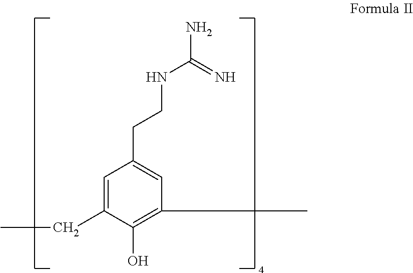

12. Combination product for use according to claim 10, in which said given antibiotic is chosen from imipenem, piperacillin-tazobactam, penicillin G, cefotaxime, ceftazidime, tobramycin, gentamicin, ciprofloxacin, rifampicin, fosfomycin, colimycin, streptomycin, ticarcilline-clavulanic acid, tigecycline or fusidic acid.

13. A method for simultaneous or separate use or spread over time for the treatment of pathologies involving at least one resistant bacterial strain belonging to a species chosen from Escherichia coli, Pseudomonas aeruginosa, and Staphylococcus aureus comprising administering to a subject in need thereof an effective amount of the combination product according to claim 10.

14. The method according to claim 13, simultaneous, separate or spread over time in the treatment of the pathologies involve a resistant bacterial strain chosen from:
a wild-type strain of Staphylococcus aureus,
a strain of meticillin-resistant Staphylococcus aureus (MRSA) without associated resistance,
a strain of MRSA having a resistance to the aminoglycosides and fluoroquinolones,
a strain of MRSA having a resistance to the aminoglycosides, fluoroquinolones, macrolides-lincosamides-synergystins and ofloxacin,
a wild-type strain of Escherichia coli,
an ESBL (extended spectrum β-Lactamase)-producing strain of Escherichia coli, having an associated resistance to the aminoglycosides, rifampicin and the trimethoprime-sulphamethoxazole combination,
a penicillinase-producing strain of Escherichia coli without associated resistance,
a cephalosporinase-hyperproducing strain of Escherichia coli having an associated resistance to the aminoglycosides, quinolones and the trimethoprime-sulphamethoxazole combination,
a wild-type strain of Pseudomonas aeruginosa,
a strain of Pseudomonas aeruginosa having a resistance to the β-lactams, the trimethoprime-sulphamethoxazole combination and fosfomycin,
a strain of Pseudomonas aeruginosa having a resistance to the β-lactams (including the carbapenems), aminoglycosides, the trimethoprime-sulphamethoxazole combination and ciprofloxacin,
a mucoid strain of Pseudomonas aeruginosa having a resistance to rifampicin and the trimethoprime-sulphamethoxazole combination.
